(19) United States
(12) Patent Application Publication
(10) Pub. No.: US 2018/0133185 A1
(43) Pub. Date: May 17, 2018

(54) Compositions and Methods for the Improvement of Memory

(71) Applicant: THE CHILDREN'S HOSPITAL OF PHILADELPHIA, Philadelphia, PA (US)

(72) Inventor: Akiva S. Cohen, Bala Cynwyd, PA (US)

(21) Appl. No.: 15/810,691

(22) Filed: Nov. 13, 2017

Related U.S. Application Data
(60) Provisional application No. 62/420,809, filed on Nov. 11, 2016.

(51) Int. Cl. A61K 31/198; A61K 31/225; A61K 47/12; A61K 47/26

(52) U.S. Cl. CPC A61K 31/198; A61K 47/26; A61K 47/12; A61K 31/225

(57) ABSTRACT

Compositions and methods are provided for the improvement of memory.

This application claims priority under 35 U.S.C. § 119(e) to U.S. Provisional Patent Application No. 62/420,809, filed on Nov. 11, 2016. The foregoing application is incorporated by reference herein.

This invention was made with government support under grant nos. NS069629 and HD059288 awarded by the National Institutes of Health. The government has certain rights in the invention.

### FIELD OF THE INVENTION

The present invention relates to the alleviation of pathology associated with brain damage, particularly traumatic brain injury.

### BACKGROUND OF THE INVENTION

Traumatic brain injury (TBI) afflicts over two million people annually in the United States, and is the primary cause of death and disability in young adults and children. TBI results in long-lasting cognitive impairment and currently there are no effective therapies to mitigate or rectify this pathology. TBI often causes enduring disabilities including emotional alterations, cognitive impairment and memory dysfunction. These functional deficits result from changes in hippocampal network excitability that are precipitated by regional imbalances between excitatory and inhibitory synaptic activity, including decreased network excitability in area CA1 and increased excitability in the dentate gyrus. Furthermore, both neuronal and astrocytic metabolism following injury are altered, including increased accumulation of lactate and an increase in intracellular glutamate, but the interaction between astrocyte-derived metabolites and neuronal metabolism following injury is not fully understood. New methods of treatment for the inhibition and/or prevention of the pathology associated with traumatic brain injury are needed.

### SUMMARY OF THE INVENTION

In accordance with the instant invention, methods for inhibiting, reducing, treating, and/or preventing in a subject the pathology associated with traumatic brain injury are provided. In a particular embodiment, the method comprises administering to the subject at least one branched chain amino acid (BCAA), particularly all three branched chain amino acids. In a particular embodiment, BCAAs are administered orally.

In accordance with another aspect of the instant invention, compositions comprising the branched chain amino acids and, optionally, at least one pharmaceutically acceptable carrier are provided. The compositions may be in an orally acceptable form such as a liquid or powder (e.g., lyophilized) form.

In accordance with another aspect of the instant invention, methods for treating a sleep disorder associated with traumatic brain injury in a subject are provided. The methods comprise administering to the subject at least one branched chain amino acid, particularly valine, leucine, and isoleucine. The branched chain amino acids may be administered in a composition with a pharmaceutically acceptable carrier, such as the oral compositions of the instant invention.

In accordance with another aspect of the instant invention, methods for improving memory function and/or reducing a cognitive impairment in a subject are provided. The methods comprise administering to the subject at least one branched chain amino acid, particularly valine, leucine, and isoleucine. The branched chain amino acids may be administered in a composition with a pharmaceutically acceptable carrier, such as the oral compositions of the instant invention. In a particular embodiment, the cognitive impairment is a memory deficiency (e.g., a deficiency in encoding). In a particular embodiment, the cognitive impairment is a deficiency in spatial (episodic) memory. In a particular embodiment, the subject has suffered a brain injury (e.g., a traumatic brain injury).

### BRIEF DESCRIPTIONS OF THE DRAWING

FIG. 1 provides a graph of freezing percentage in a conditioned fear response (CFR) assay of mice receiving BCAA diet (treatment) or receiving placebo without the BCAA diet (placebo) for 2, 3, 4, or 5 days after fluid percussion injury.

FIG. 2 provides a graph of freezing percentage in a CFR assay of mice receiving BCAA diet for 10 days (treatment), brain injured animals without the BCAA diet for 10 days (placebo), or on the BCAA diet for 5 days and then without for 5 days (5 day +/5 day −) after fluid percussion injury.

FIG. 3 provides a graph of freezing percentage in a CFR assay of mice receiving BCAA diet at 100 mM or 50 mM (treatment) or controls without the BCAA diet (placebo) for 5 days after fluid percussion injury.

FIG. 4 provides a graph of freezing percentage in a CFR assay of mice receiving BCAA diet via gavage (treatment) or controls without the BCAA diet (placebo) for 3 or 5 days after fluid percussion injury.

FIG. 5 provides a graph of freezing percentage in a CFR assay of sham mice, mice receiving glyceryl triacetate (GTA) via gavage after injury (FPI acetate) or controls without the GTA diet after injury (FPI) for 5 days after fluid percussion injury. * P<0.05.

FIG. 6A provides the activity monitoring timeline. Activity patterns were binned into post-op, acute, subacute and chronic time points following injury or sham surgery. FIG. 6B shows the percent time spent active during the dark phase is decreased across acute, subacute and chronic periods after TBI. The average length of each inactivity bout did not significantly differ between groups (FIG. 6C). However, the average length of each activity bout was significantly decreased after TBI across the three time points (FIG. 6D). Active bouts longer than 30 minutes were particularly affected after TBI across all three phases (FIG. 6E). The number of transitions from active to inactive bouts was significantly increased after TBI across the three time points (FIG. 6F). + p<0.1, *p<0.05.

FIG. 7 provides the experimental timeline for EEG/EMG recording.

FIGS. 8A-8D show that baseline EEG recordings over 24 hours show significant alterations in wake and sleep patterns, which are ameliorated by BCAA dietary intervention. TBI mice spend less time awake and more time in NREM sleep (FIG. 8A). FIG. 8B provides representative hypnograms from Sham, TBI and TBI+BCAA mice. Note the absence of the long wake period at 19:00 (Lights Off) in the TBI mouse that is restored in TBI+BCAA mouse. FIG. 8C provides the distribution of wake bout length over the circadian cycle shows that TBI significantly shortens wake bouts throughout the light and dark phases, and the normal diurnal fluctuation in wake bout length is abolished. FIG. 8D provides the distribution of sleep (NREM+REM) bout length over the circadian cycle shows significantly shorter sleep bouts in TBI mice during the dark phase. ZT=Zeitgeber Time; ZTO-3=7:00 AM-10:00 AM, ZT13−15=7:00 PM-10:00 PM, and so forth. *p<0.05, **p<0.01, ***p<0.001.

FIGS. 9A-9E show that TBI causes significant behavioral state instability, which is restored by BCAA therapy. TBI significantly increases the total number of wake to sleep (NREM+REM) transitions during the dark, or active phase (FIG. 9A). FIG. 9B shows the transitions sub-categorized by Wake to NREM (WN), NREM to Wake (NW), NREM to REM (NR), REM to NREM (RN), and REM to Wake (RW) during the light phase. FIG. 9C shows the transitions subcategories during the dark phase. Note TBI mice have more transitions to and from Wake (WN, NW, RW), and this is restored by BCAA therapy. Also, group differences are more robust in the dark phase compared to the light phase. TBI mice have significantly more short wake bouts compared to Sham mice, and BCAA therapy restores the distribution of long wake bouts. FIGS. 9D and 9E show that group differences in wake bout lengths are more robust in the dark phase compared to the light phase. + p<0.1, *p<0.05.

FIG. 10A provides wake power spectra. Statistical group differences denoted as a black bar above the power spectral curves from 7 to 24 Hz. Note the significant left shift in theta peak frequency (TPF) after TBI. FIG. 10B provides NREM power spectra. Group differences are denoted as a black bar above the power spectral curves from 7 to 24 Hz. FIG. 10C provides REM power spectra. Group differences are denoted as a black bar above the power spectral curves again ranging from 7-24 Hz. Again, there is a significant left shift in TPF after TBI. Thin black bar, p<0.10, medium black bar, p<0.05, thick black bar, p<0.01; **p<0.01.

FIG. 11A shows that after being placed in novel environment, TBI mice have a shorter latency to the first sleep episode which is reinstated with BCAA therapy. FIG. 11B shows the percentage of epochs scored as wake across 5 minute bins was calculated, and shows that TBI mice have less wake epochs throughout the 2 hour challenge. BCAA therapy restores wakefulness during the novel environment challenge. +p<0.10.

FIGS. 12A-12D show TBI mice show a higher sleep pressure after sleep deprivation. After a relatively short 3 hour period of sleep deprivation, TBI mice spend more time in NREM sleep during the recovery period. BCAA therapy decreases NREM sleep to Sham levels (FIG. 12A). BCAA therapy also restored NREM bout length to sham levels (FIG. 12B). FIG. 12C shows the delta power, indicating a higher sleep pressure, during recovery sleep. FIG. 12D shows that TBI mice also have a lower percentage of wakefulness during the recovery period after sleep deprivation. +p<0.10, *p<0.05, **p<0.01.

FIG. 13A provides a schematic coronal section from mouse brain showing the lateral hypothalamus (LH, left box), the region where orexin neurons reside and where orexin cells were counted. The right box represents the area depicted in photomicrographs in FIGS. 13D, 13E, and 13F. FIG. 13B provides a photomicrograph of LH showing presynaptic glutamate vesicles (VGLUT1 immunolabeling) in close proximity to orexin cell bodies (orexin-A immunolabeling) at 40×. FIG. 13C provides the quantification of Fos-positive orexin neurons, which are a marker of neural activation after the 3 hour wake challenge, showing significantly decreased orexin activation after TBI compared to Sham mice, which is restored with BCAA therapy (F=22.47, p<0.0001; TBI v. Sham p<0.001, TBI v. TBI+BCAA p<0.01; one-way ANOVA followed by Dunnett's post-hoc tests). FIGS. 13D, 13E, and 13F provide representative photos of LH from Sham, TBI, and TBI+BCAA mice showing orexin colocalization with Fos at 20×. f=fornix. Scale bar=50 um. **p<0.01, ***p<0.001.

FIG. 14 provides a schematic of the spatial object recognition task which consists of 3 different phases interlaid by resting time. In the first phase (Habituation), animals explored an empty apparatus. In the second phase (Familiarization) animals were exposed to two identical objects (filled circles) with 1-hour interphase rest. In the third phase (Test), one object was displayed in a previous empty quadrant and the animal entered the field from a different starting point. Between familiarization 2 and test the delay interval was randomly chosen between 3 minutes, 1 hour and 24 hours. Each animal performed the task with one delay time.

FIG. 15 provides a graph of memory retrieval. Novelty preference was assessed in sham and TBI condition during performance the test after 3 minutes, 1 hour and 24 hours. There was no difference in performance between TBI and Sham animals when discriminating the new location when tested after 3-minutes delay. However, after 1 hour animals in the TBI condition had a decrease in performance compared to SHAM animals (asterisks in plot, T₍₁₈₎=2.36, p<0.05). Bars shows mean and error bars show SEM.

FIG. 16 shows the exploratory behavior (EB) and discriminatory index (DI) of TBI and Sham mice as analyzed during the first (F1) and second (F2) rounds of familiarization and the test.

FIGS. 17A-17F show memory encoding and exploration time during first familiarization. FIG. 17A shows the entire behavioral data for all animals (TBI and sham) during encoding (familiarization 1) and FIG. 17D shows the entire behavioral data for all animals (TBI and sham) during familiarization 2. Each row represents an animal (TBI, N=29; Sham, N=29). The lines in each row represent time windows in which the mouse was interacting with the objects. Light colors and dark colors represent one of object 1 and object 2, respectively. FIGS. 17B and 17E show the data in FIGS. 17A and 17D, respectively, segmented into thirty-four 90 second time windows incremented by 15 second intervals. The percentage of time spent interacting (percent interaction) with the objects was recorded for each animal for each time window. The percentage interaction was then averaged across animals separately for TBI and sham animals. Error bars represent S.E.M. For FIGS. 17C and 17F, the time periods were simplified to statistically analyze the data in to three non-overlapping windows of 200 seconds duration. The percent interaction was calculated in each time window for all animals and a 2-way ANOVA was performed with time and condition as factors. There was a main effect across time (F_{2,198}=26.32, p<0.001) and an interaction effect between time and condition (F_{4,198}=2.59, p<0.05) that was driven by an increase in exploration time for SHAM mice in the first time window (* p<0.03). Error bars represent S.E.M.

FIGS. 18A and 18B show the effect of BCAA therapy on memory encoding and retrieval. FIG. 18A shows the analysis performed for FIG. 14C, but with a cohort of animals that were fed a BCAA rich diet starting 48 hours after injury. The BCAA mice experience a high rate of object exploration during the encoding period of the task. Two-way ANOVA shows main effect across time (F_{(2,198)}=26.32, p<0.001) and an interaction effect between time and TBI vs Sham vs BCAA condition (F_{(4,198)}=2.59, p<0.05). These results were driven by a recovery of exploration in the TBI group after therapy (TBI vs TBI+BCAA post-hoc t-test, **T₍₃₈₎=−0.69, p<0.01). FIG. 18B shows the novelty preference assessed during test phase in sham (n=10), TBI (n=10) and TBI+BCAA (n=11) after 1-hour delay between familiarization 1 and test. BCAA therapy showed an improvement in the task performance (**T=18, p<0.05, *T₍₁₉₎=1.96, p=0.06). Bars shows mean and error bars show SEM.

FIGS. 19A-19D provide memory models to explain memory deficits after TBI. FIG. 19A: The behavioral results can be understood in terms of strength model of memory. In this case there are three hypotheses that could explain the data, FIG. 19B: a deficit of encoding, FIG. 19C: a deficit of maintenance, and FIG. 19D: a deficit of retrieval. The data presented are most consistent with a deficit of encoding.

FIGS. 20A-20C provide schematic representation of broadband correction. FIG. 20 A provides a schematic of the averaged power spectrum of one animal in the sham condition. FIG. 20B provides a schematic of the distribution of power values across all time epochs. The line represents a regression through the distribution of power values. The broadband power is represented by the height of the vertical line. The residuals (regression line subtracted from the power values) represent the broadband correction. FIG. 20C provide a schematic of the broadband corrected power spectra for the same sham animal.

FIGS. 21A and 21B provide graphs of the averaged total broadband power in sham and TBI animals segmented into 120 second time windows during the first familiarization in area CA1 and CA3, respectively. FIGS. 21C and 21D provide graphs of the averaged broadband corrected residual theta power in sham and TBI animals segmented into 120 second time windows with 5% overlap during the first familiarization in area CA1 and area CA3, respectively. Error bars represent S.E.M. * p value<0.05.

FIG. 22 provides graphs of averaged total broadband power and averaged broadband corrected residual theta power in sham and TBI in area CA3 and CA1 during the exploration objects time windows. Error bars represent S.E.M.

FIG. 23A provides a graph of the discrimination index of both the sham and injured groups. FIG. 23B shows the discrimination index for sham and injured groups separately to show variance. FIG. 23C provides a graph of the discrimination indexes for sham, LFPI and BCAA groups. All p values were statistically significant (P value<0.001***).

### DETAILED DESCRIPTION OF THE INVENTION

Studies in mice have demonstrated that the hippocampus, a brain structure involved in learning and memory and often injured in concussion, has reduced levels of the three branched chain amino acids (BCAAs) valine, isoleucine and leucine after traumatic brain injury. Dietary administration of BCAAs restored the concentrations of these amino acids in the injured hippocampus, and restored cognitive function to levels not significantly different to non-injured animals.

Dietary BCAA administration has been studied extensively in healthy people and in a variety of disease states over many decades (Fernstrom, J. D. (2005) J. Nutr., 135:1539S-1546S). BCAA therapy has been studied as it relates to exercise physiology, hepatic pathology, and various neurological and psychiatric disorders (Richardson et al. (2003) Am. J. Psychiatry 160:1117-1124; Richardson et al. (1999) Psychopharmacol., 143:358-364; Tandan et al. (1996) Neurology 47:1220-1226; Mori et al. (2002) J. Neurol. Sci., 195:149-152; Mori et al. (1999) Intern. Med., 38:401-406; Scarna et al. (2003) Br. J. Psychiatry 182:210-213). Patients have been treated with BCAA's for variable lengths of time including chronically for more than 2 years (Muto et al. (2005) Clin. Gastroenterol. Hepatol., 3:705-713). BCAA's have been given orally and as an intravenous infusion (Aquilani et al. (2005) Arch. Phys. Med. Rehabil., 86:1729-1735; Aquilani et al. (2008) Arch. Phys. Med. Rehab., 89:1642-1647; Kirvela et al. (1998) Pharmacol. Biochem. Behav., 60:77-82). The dosing used in these previous studies ranges from a few grams per day to over 100 g/d (Tangkijvanich et al. (2000) Southeast Asian J. Trop. Med. Public Health 31:152-157; Kuroda et al. (2010) J. Gastroenterol. Hepatol., 25:1550-1555). For example, oral doses of 60 g/d have been shown to be effective in reducing mania symptoms (Scarna et al. (2003) Br. J. Psychiatry 182:210-213). Benefit has also been seen in tardive dyskinesia (˜15 g/d), spinocerebellar degeneration (6 g/d), anorexia in cancer patients (14 g/d), and hepatic encephalopathy (median amount of BCAA over 11 randomized trials was 28 g/d, with the range of 11 to 57 g/d) (Richardson et al. (2003) Am. J. Psychiatry 160:1117-1124; Richardson et al. (1999) Psychopharmacol., 143:358-364; Mori et al. (2002) J. Neurol. Sci., 195:149-152; Mori et al. (1999) Intern Med., 38:401-406; Cangiano et al. (1996) J. Natl. Cancer Inst., 88:550-552; Als-Nielsen et al. (2003) Cochrane Database Syst. Rev., 2003:CD001939).

Several review papers have examined these studies and summarized the infrequent adverse events (Als-Nielsen et al. (2003) Cochrane Database Syst. Rev., 2003:CD001939; Fernstrom, J. D. (2005) J. Nutr., 135:1539S-1546S). Overall, BCAA's are well tolerated and are associated with minimal to no side effects. There are reports of mild gastrointestinal side effects such as abdominal distention, diarrhea, and constipation. No serious or life threatening side effects have been reported. These studies indicate that humans can consume BCAA's in considerable amounts without adverse effects and, in some cases, with significant benefit to the study populations. Indeed, athletes already consume BCAA's as over the counter nutritional supplements to augment their exercise program.

There are several clinical studies of BCAA use in children. Studies to determine the mean BCAA requirement in healthy school age children and children with mild-to-moderate chronic cholestatic liver disease were conducted by giving children varying amounts of BCAA's (Mager et al. (2003) J. Nutr., 133:3540-3545; Mager et al. (2006) J. Nutr., 136:133-139). BCAA's have been administered safely to children with phenylketonuria in an attempt to inhibit entry of phenylalanine into the brain and reduce its toxic effects on the central nervous system (Berry et al. (1982) Ped. Res., 16:751-755; Berry et al. (1990) Am. J. Dis. Child, 144:539-543; Jordan et al. (1985) Dev. Med. Child Neurol., 27:33-39). Beneficial effects were also seen when children with end-stage liver disease awaiting transplantation were fed with a BCAA-enriched formula compared with a standard formula (Chin et al. (1992) Amer. J. Clin. Nutr., 56:158-163; Chin et al. (1990) J. Gastroenterol. Hepatol., 5:566-572). Symptoms of tardive dyskinesia were reduced in children and adolescents after treatment with BCAA's, and epileptic children treated with up to 20 g/d of BCAA's in conjunction with a ketogenic diet showed reduction in seizure frequency and improvement in behavior and cognitive functioning (Richardson et al. (2004) J. Clin. Psychiatry 65:92-96; Evangeliou et al. (2009) J. Child Neurology 24:1268-1272). In all studies of children and adolescents treated with BCAA's, the supplements were well tolerated and without side effects.

A limited number of studies have examined the efficacy of BCAA's in humans after TBI. One study evaluated plasma amino acid concentrations in patients admitted to a rehabilitation facility approximately two months after TBI and found significantly reduced levels of BCAA's, among other amino acids, relative to age-matched, non-injured controls (Aquilani et al. (2000) Arch. Phys. Med. Rehab., 81:176-181). A follow-up study on a subsequent cohort of TBI patients found that plasma concentrations of amino acids were still reduced 120 days after injury, mainly driven by lower valine levels (Aquilani et al. (2003) Arch. Phys. Med. Rehab., 84:1258-1265). Work by the same group demonstrated that intravenous BCAA's administered to patients in the rehabilitation stage after severe TBI improved disability rating scale scores when compared to placebo (Aquilani et al. (2005) Arch. Phys. Med. Rehab., 86:1729-1735). Additionally, they showed that BCAA's might improve recovery from a post-traumatic vegetative or minimally conscious state (Aquilani et al. (2008) Arch. Phys. Med. Rehab., 89:1642-1647).

The instant invention provides a BCAA rich composition, particularly a liquid consumable, for treatment of cognitive impairment associated with concussion. In a particular embodiment, the composition comprises valine, leucine, and isoleucine in approximately equivalent amounts (e.g., a 1:1:1 ratio). The amount of BCAA may be determined, for example, by weight or molar amount. While the BCAAs will often be present in equivalent amounts, the compositions of the instant invention may comprise excess amounts of one or two of the BCAAs. For example, the composition may comprise up to ten times, up to five times, or up to three times excess of one or two amino acids branched amino acid compared to another. For example, the composition may comprise excess valine.

Branched-chain amino acids are amino acids that have a fork or branch in the side chain. Branched chain amino acids include leucine, isoleucine and valine and precursors or analogs thereof. BCAAs may be administered in their free forms or salts thereof, as dipeptides, tripeptides, polypeptides (e.g., from about 2 to about 10 amino acids), and/or BCAA-rich proteins (e.g., proteins comprising at least 25%, at least 50%, or at least 75% or more BCAA content). In a particular embodiment, dipeptides, tripeptides and polypeptides may include two or more BCAAs. Where non-BCAAs are included in a dipeptide, tripeptide, or polypeptide, the non-BCAAs may be any amino acid, particularly alanine and/or glycine. Examples of dipeptides include, without limitation: isoleucyl-leucine, leucyl-alanine, alanyl-leucine, alanyl-isoleucine, alanyl-valine, glycyl-leucine, glycyl-isoleucine, and glycyl-valine.

Leucine precursors, such as pyruvate, and metabolites, such as β-hydroxy-β-methylbutyrate and α-ketoisocaproate, exhibit properties similar to those of leucine. These compounds may be administered as BCAAs as they are converted into the above-mentioned BCAA in vivo.

In a particular embodiment, the composition comprises independently from about 1 mg/ml to about 50 mg/ml, from about 5 mg/ml to about 25 mg/ml, from about 10 mg/ml to about 20 mg/ml, from about 13 mg/ml to about 20 mg/ml, or about 16 mg/ml of each of the three BCAA.

In a particular embodiment, at least about 40 g, at least about 50 g, at least about 60 g, at least about 70 g, or more of BCAAs are administered to the subject per day. In a particular embodiment, about 40 g to about 100 g of BCAAs are administered daily, particularly about 60 g to about 100 g, about 60 g to about 75 g, or about 60 g. Taking the subject's weight into account, at least about 40 g/70 kg, at least about 50 g/70 kg, at least about 60 g/70 kg, at least about 70 g/70 kg, or more of BCAAs are administered to the subject per day. In a particular embodiment, about 40 g/70 kg to about 100 g/70 kg of BCAAs are administered daily, particularly about 60 g/70 kg to about 100 g/70 kg, about 60 g/70 kg to about 75 g/70 kg, or about 60 g/70 kg. The BCAAs may be administered in more than one dosage to reach the daily goal (e.g., administered twice, three times, four times or more daily).

Compositions comprising large quantities of branched chain amino acids are not palatable to many subjects, particularly humans. Accordingly, compositions of the instant invention comprise at least one flavor masking agent, particularly a bitter taste receptor blocker. In a particular embodiment, the flavor masking agent is a salt of an organic acid. Examples of organic acids include, without limitation, gluconic acid and lactic acid. Suitable salts include, by way of example, calcium lactate, magnesium lactate, sodium lactate, calcium gluconate, magnesium gluconate, and sodium gluconate. In particular embodiment, the flavor masking agent is sodium gluconate.

In a particular embodiment, the flavor masking agent is present in the composition at about 5 g, at about 10 g, at about 15 g, at about 20 g, or more per 30 g of BCAAs. In a particular embodiment, the composition comprises at least about 50 mM, at least about 75 mM, at least about 100 mM, or more of the flavor masking agent. The compositions may also comprise at least one sweetener. Any natural or artificial sweetener may be used. Examples of sweeteners include, without limitation, sucrose, fructose, maltose, dextrose, sucralose, aspartame, saccharin, and the like. In a particular embodiment, the sweetener is sucralose.

In a particular embodiment, the sweetener is present in the composition at about 2 g, at about 5 g, at about 10 g, at about 15 g, or more per 30 g of BCAAs. In a particular embodiment, the composition comprises at least about 10 mM, at least about 20 mM, at least about 35 mM, or more of the sweetener.

The compositions may also comprise at least one flavoring agent. Any natural or synthetic flavor agent can be used in the present invention. One or more flavoring agents may be used to enhance the palatability of the compositions. The flavoring agent may be an emulsion, concentrate, aqueous- or oil-soluble liquid, dry powder, or a combination thereof. In a particular embodiment, the flavor agent is a concentrate or powder. In a particular embodiment, the flavoring agent is a fruit flavor. In a particular embodiment, the flavor agent is a concentrate powder (e.g., unsweetened tropical punch KoolAid® powder).

The methods of the instant invention may further comprise the administration of acetate. The compositions of the instant invention may also comprise acetate. Acetate may be administered to the subject as acetic acid or a pharmaceutically acceptable salt thereof, such as calcium acetate. In a particular embodiment, the acetate is delivered in a hydrophobic form, such as glyceryl triacetate (GTA; the acetate triester of glycerol). It has been shown that GTA can be used as a dietary supplement to increase acetate levels in the brain by over 15-fold within one hour of administration (Mathew et al. (2005) J. Pharmacol. Exp. Ther., 315:297-303). In a particular embodiment, about 0.5 mg/kg to about 100 mg/kg of GTA are administered daily, particularly about 0.5 mg/kg to about 10 mg/kg, about 2.5 mg/kg to about 7.5 mg/kg, or about 5 mg/kg.

In a particular embodiment, the composition of the instant invention comprises 10 g valine, 10 g leucine, 10 g isoleucine, 13 g sodium gluconate, 9 g sucralose, and a flavor concentrate in 630 ml of water. The composition may further comprise acetate (e.g., 175 mg GTA). The compositions of the instant invention may have the same ratio of components (+5%) in different volumes of water.

As used herein, “traumatic brain injury” or “TBI” refers to an acquired brain injury or a head injury, when a trauma causes damage to the brain. Trauma includes, e.g., post-head trauma, impact trauma, and other traumas to the head such as, for example, traumas caused by accidents and/or sports injuries, concussive injuries, penetrating head wounds, brain tumors, stroke, heart attack, meningitis, viral encephalitis, and other conditions that deprive the brain of oxygen. In a preferred embodiment, the trauma is an external, physical force.

The damage can be focal (confined to one area of the brain) or diffuse (involving more than one area of the brain). Clinically, traumatic brain injury can be rated as mild, moderate or severe based on TBI variables that include duration of loss of consciousness (LOC), Glasgow Coma Score (GCS; e.g., mild 13-15; moderate=9-12; severe=<8) and post traumatic stress amnesia (see, e.g., Levin et al. (1979) J. Nervous Mental Dis., 167:675-84; Holm et al. (2005) J. Rehabil. Med., 37:137-41). In a particular embodiment, the TBI is mild or moderate.

In some embodiments, the traumatic brain injury can be chronic, where the brain is subject to repeated traumatic injury to the brain. Generally, chronic traumatic brain injury is typically a mild to moderate form of closed brain injury repeatedly suffered by a subject (e.g., athlete), resulting in increased incidence of impaired motor, cognitive, and/or behavioral impairments months to years following the traumatic brain injuring events. Individuals subjected to such chronic brain injury appear to have increased susceptibility to certain neurological disorders, such as Alzheimer's disease, chronic traumatic encephalopathy (CTE), and/or Parkinson's Disease.

In some embodiments, the traumatic brain injury can result from a closed head injury. The closed head injury may be transient or prolonged. A “closed head injury” refers to a brain injury when the head suddenly and violently hits an object but the object does not break through the skull. In some embodiments, the closed head injury is a concussion or contusion. A concussion is a mild form of traumatic brain injury resulting in temporary impairment of neurological function which quickly resolves by itself, and where there are generally no gross structural changes to the brain as the result of the condition. A contusion is a distinct area of swollen brain tissue mixed with blood released from broken blood vessels. A contusion can also occur in response to shaking of the brain back and forth within the confines of the skull, an injury referred to as “contrecoup.” As used herein, a closed head injury refers to an injury due to an external, physical trauma and does not encompass brain injury resulting from “internal” forces such as ischemia/reperfusion and stroke.

Sleep disorders are a common pathology associated with traumatic brain injury and can significantly impair cognitive rehabilitation. Hereinbelow, a well-established mouse model of mild brain injury (i.e., lateral fluid percussion injury) was used which recapitulates the chronic sleep disturbances seen in the human condition. Brain-injured mice demonstrate a persistent inability to maintain wakefulness and severe sleep-wake fragmentation. EEG power spectral analyses show a shift to slower peak theta frequencies. When placed in a novel environment, injured mice have a shorter latency and higher pressure to sleep. TBI-induced sleep disturbance resembles the narcoleptic phenotype, and the neuropeptide orexin (also known as hypocretin) has been implicated in both disorders. Brain-injured mice show significantly less activation of orexin neurons in response to sustained wakefulness. To ameliorate TBI-induced pathology including orexin neuron activation, mice were given a dietary supplement consisting of branched chain amino acids (BCAA) which are precursors for de novo glutamate synthesis in the brain. BCAA therapy reinstated activation of orexin neurons in injured mice and restored wakefulness by increasing wake time, consolidating sleep and wake bouts, and increasing arousal during wake challenges. The data identify novel mechanisms underlying sleep disturbances in a model of mild TBI and indicate that BCAA intervention, likely acting in part through orexin, can normalize injury-induced sleep disturbances and, thus, facilitate cognitive rehabilitation.

In accordance with another aspect of the instant invention, methods for treating, preventing, and/or inhibiting a sleep disorder associated with a traumatic brain injury are provided. The methods comprise administering branched chain amino acids to a subject who has sustained a traumatic brain injury. The sleep disorders associated with a traumatic brain injury are generally known in the art (see, e.g., Kushner, D. (1998) Arch. Intern. Med., 158:1617-24; Parsons et al. (1982) Nurs. Res., 31:260-264; Viola-Saltzman et al. (2012) Neurol. Clin., 30:1299-312; Castriotta et al. (2011) CNS Drugs 25:175-85). In a particular embodiment, the sleep disorder is a problem/disruption with the onset and/or maintenance of sleep (e.g., sleep-wake fragmentation). Examples of sleep disorders associated with a traumatic brain injury include, without limitation, insomnia (e.g., difficulty with falling asleep and/or staying asleep), fatigue, drowsiness, sleep apnea, narcolepsy, and/or sleepiness (e.g., excessive daytime sleepiness). Sleep disorders may be diagnosed any means such as a sleep study, polysomnography, multiple sleep latency testing, maintenance of wakefulness testing, and/or actigraphy. The methods of the instant invention may further comprise examining/diagnosing the patient for a sleep disorder after the traumatic brain injury and prior to treatment. The methods may also further comprise examining/diagnosing the patient for a sleep disorder after the traumatic brain injury and after treatment. Treatment with the BCAAs may be stopped once the sleep disorder has been resolved.

As stated above, the methods comprise administering branched chain amino acids to a subject who has sustained a traumatic brain injury. The BCAAs may be administered before, during, and/or after the traumatic brain injury. For example, the subject may be administered the BCAAs prior to sustaining a traumatic brain injury (e.g., prior to participating in an activity with an increased likelihood of sustaining a traumatic brain injury such as participating in a contact sport (e.g., hockey, football, rugby, etc.). The subject may be administered BCAA therapy after sustaining the injury. In a particular embodiment, the subject is administered BCAA therapy (e.g., daily) prior to sleep (e.g., within about 30 minutes or about 1 hour).

The BCAAs may be administered to the subject by any means. In a particular embodiment, the BCAAs are administered in a liquid consumable (e.g., the palatable/drinkable compositions described hereinabove). The BCAAs administered to the subject may include one, two, or all three of valine, leucine, and isoleucine. In a particular embodiment, valine, leucine, and isoleucine are administered in approximately equivalent amounts (e.g., a 1:1:1 ratio). The amount of BCAA may be determined, for example, by weight or molar amount. While the BCAAs will often be administered in equivalent amounts, excess amounts of one or two of the BCAAs may be administered. For example, up to ten times, up to five times, or up to three times excess of one or two amino acids compared to another may be administered. For example, the excess valine compared to leucine and/or isoleucine may be administered.

As explained hereinabove, the BCAAs may be administered in their free forms or salts thereof, as dipeptides, tripeptides, polypeptides (e.g., from about 2 to about 10 amino acids), and/or BCAA-rich proteins (e.g., proteins comprising at least 25%, at least 50%, or at least 75% or more BCAA content). The BCAAs may be isolated. In a particular embodiment, dipeptides, tripeptides and polypeptides may include two or more BCAAs. Where non-BCAAs are included in a dipeptide, tripeptide, or polypeptide, the non-BCAAs may be any amino acid, particularly alanine and/or glycine. Examples of dipeptides include, without limitation: isoleucyl-leucine, leucyl-alanine, alanyl-leucine, alanyl-isoleucine, alanyl-valine, glycyl-leucine, glycyl-isoleucine, and glycyl-valine.

Leucine precursors, such as pyruvate, and metabolites, such as β-hydroxy-β-methylbutyrate and α-ketoisocaproate, exhibit properties similar to those of leucine. These compounds may be administered as BCAAs as they are converted into the above-mentioned BCAA in vivo.

In a particular embodiment, at least about 40 g, at least about 50 g, at least about 60 g, at least about 70 g, or more of BCAAs are administered to the subject per day. In a particular embodiment, about 40 g to about 100 g of BCAAs are administered daily, particularly about 60 g to about 100 g, about 60 g to about 75 g, or about 60 g. Taking the subject's weight into account, at least about 40 g/70 kg, at least about 50 g/70 kg, at least about 60 g/70 kg, at least about 70 g/70 kg, or more of BCAAs are administered to the subject per day. In a particular embodiment, about 40 g/70 kg to about 100 g/70 kg of BCAAs are administered daily, particularly about 60 g/70 kg to about 100 g/70 kg, about 60 g/70 kg to about 75 g/70 kg, or about 60 g/70 kg. The BCAAs may be administered in more than one dosage to reach the daily goal (e.g., administered twice, three times, four times or more daily).

In a particular embodiment, the BCAAs are administered immediately or soon after the traumatic brain injury event. For example, the BCAAs are administered at least within a month of injury, within two weeks of injury, within about the first 2, 3, 4 or 7 days after injury, within about the first day after injury, or within about the first hour after injury. In a particular embodiment, the BCAAs are administered within about the first 2 days of the injury. The BCAAs may be administered continually (e.g., every day) after the injury for at least one week, particularly at least two weeks, at least three weeks, at least four weeks or more.

In accordance with another aspect of the instant invention, methods for decreasing cognitive impairment in a subject are provided. In a particular embodiment, the method improves the memory of the subject and/or reduces memory deficiency in the subject. In a particular embodiment, the method reduces and/or inhibits memory loss and/or deficiency, reduces and/or inhibits a decreased ability to form short- or long-term memory, reduces and/or inhibits decreased episodic memory, and/or reduces and/or inhibits decreased encoding. The methods comprise administering branched chain amino acids to a subject in need thereof. In a particular embodiment, the subject has sustained a traumatic brain injury (e.g., a mild traumatic brain injury). In a particular embodiment, the subject has a disease, disorder, or condition which causes or is characterized by or is associated with a memory deficiency (e.g., as set forth above). In a particular embodiment, the subject has a disease, disorder, or condition, which causes or is characterized by or associated with a memory deficiency such as decreased or deficient episodic memory and/or decreased or deficient encoding. Examples of such diseases, disorders, or conditions include, without limitation, aging, Alzheimer's disease, Parkinson's disease, Lewy-bodies dementia, dementia, chronic traumatic encephalopathy (CTE), dyslexia, learning disability, attention-deficit/hyperactivity disorder (ADHD), stroke, traumatic brain injury, asphyxia (e.g., perinatal asphyxia, cerebral asphyxia), cardiac arrest, coma, meningitis, and epilepsy and status epilepticus. The methods of the instant invention may further comprise examining/diagnosing the patient for such a disease, disorder, or condition and/or memory function prior to treatment. The methods may also further comprise examining/diagnosing the patient for memory function after treatment. Treatment with the BCAAs may be stopped once the memory function has been resolved.

As stated above, the methods comprise administering branched chain amino acids to a subject to improve the memory of the subject and/or reduce a memory deficiency or cognitive impairment in the subject. When the memory deficiency or cognitive impairment is caused by a traumatic brain injury, the BCAAs may be administered before, during, and/or after the traumatic brain injury. For example, the subject may be administered the BCAAs prior to sustaining a traumatic brain injury (e.g., prior to participating in an activity with an increased likelihood of sustaining a traumatic brain injury such as participating in a contact sport (e.g., hockey, football, rugby, etc.). The subject may be administered BCAA therapy after sustaining the injury. In a particular embodiment, the subject is administered BCAA therapy after the injury.

The BCAAs may be administered to the subject by any means. In a particular embodiment, the BCAAs are administered in a liquid consumable (e.g., the palatable/drinkable compositions described hereinabove). The BCAAs administered to the subject may include one, two, or all three of valine, leucine, and isoleucine. In a particular embodiment, valine, leucine, and isoleucine are administered in approximately equivalent amounts (e.g., a 1:1:1 ratio). The amount of BCAA may be determined, for example, by weight or molar amount. While the BCAAs will often be administered in equivalent amounts, excess amounts of one or two of the BCAAs may be administered. For example, up to ten times, up to five times, or up to three times excess of one or two amino acids compared to another may be administered. For example, the excess valine compared to leucine and/or isoleucine may be administered.

As explained hereinabove, the BCAAs may be administered in their free forms or salts thereof, as dipeptides, tripeptides, polypeptides (e.g., from about 2 to about 10 amino acids), and/or BCAA-rich proteins (e.g., proteins comprising at least 25%, at least 50%, or at least 75% or more BCAA content). The BCAAs may be isolated. In a particular embodiment, dipeptides, tripeptides and polypeptides may include two or more BCAAs. Where non-BCAAs are included in a dipeptide, tripeptide, or polypeptide, the non-BCAAs may be any amino acid, particularly alanine and/or glycine. Examples of dipeptides include, without limitation: isoleucyl-leucine, leucyl-alanine, alanyl-leucine, alanyl-isoleucine, alanyl-valine, glycyl-leucine, glycyl-isoleucine, and glycyl-valine.

Leucine precursors, such as pyruvate, and metabolites, such as β-hydroxy-β-methylbutyrate and α-ketoisocaproate, exhibit properties similar to those of leucine. These compounds may be administered as BCAAs as they are converted into the above-mentioned BCAA in vivo.

In a particular embodiment, at least about 40 g, at least about 50 g, at least about 60 g, at least about 70 g, or more of BCAAs are administered to the subject per day. In a particular embodiment, about 40 g to about 100 g of BCAAs are administered daily, particularly about 60 g to about 100 g, about 60 g to about 75 g, or about 60 g. Taking the subject's weight into account, at least about 40 g/70 kg, at least about 50 g/70 kg, at least about 60 g/70 kg, at least about 70 g/70 kg, or more of BCAAs are administered to the subject per day. In a particular embodiment, about 40 g/70 kg to about 100 g/70 kg of BCAAs are administered daily, particularly about 60 g/70 kg to about 100 g/70 kg, about 60 g/70 kg to about 75 g/70 kg, or about 60 g/70 kg. The BCAAs may be administered in more than one dosage to reach the daily goal (e.g., administered twice, three times, four times or more daily).

In a particular embodiment, the BCAAs are administered immediately or soon after the event associated with the disease, disorder, or condition (e.g., traumatic brain injury). For example, the BCAAs are administered at least within a month of the event or injury, within two weeks of the event or injury, within about the first 2, 3, 4 or 7 days after the event or injury, within about the first day after the event or injury, or within about the first hour after the event or injury. In a particular embodiment, the BCAAs are administered within about the first 2 days of the event or injury. The BCAAs may be administered continually (e.g., every day) after the injury for at least one week, particularly at least two weeks, at least three weeks, at least four weeks or more.

### I. Definitions

The singular forms “a,” “an,” and “the” include plural referents unless the context clearly dictates otherwise.

As used herein, “cognitive impairment” refers to an acquired deficit in at least one of the following: memory function, problem solving, orientation, and abstraction. The deficiency typically impinges on an individual's ability to function independently.

The term “substantially pure” refers to a preparation comprising at least 50-60% by weight of a given material (e.g., nucleic acid, oligonucleotide, protein, etc.). More preferably, the preparation comprises at least 75% by weight, and most preferably 90-95% by weight of the given compound. Purity is measured by methods appropriate for the given compound (e.g., chromatographic methods, agarose or polyacrylamide gel electrophoresis, HPLC analysis, and the like).

The terms “isolated” is not meant to exclude artificial or synthetic mixtures with other compounds or materials, or the presence of impurities that do not interfere with the fundamental activity, and that may be present, for example, due to incomplete purification, or the addition of stabilizers.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, preservative, antioxidant, solubilizer, emulsifier, adjuvant, excipient, bulking substances, auxiliary agent or vehicle with which an active agent of the present invention is administered. Pharmaceutically acceptable carriers can be sterile liquids, such as water and oils, including those of petroleum, animal, vegetable or synthetic origin, such as peanut oil, soybean oil, mineral oil, sesame oil and the like. Water or aqueous saline solutions and aqueous dextrose and glycerol solutions are preferably employed as carriers, particularly for injectable solutions. Suitable pharmaceutical carriers are described, for example, in “Remington's Pharmaceutical Sciences” by E. W. Martin.

The term “pathology” refers to any deviation from a healthy or normal condition, such as a disease, disorder, syndrome, or any abnormal medical condition.

The term “treat” as used herein refers to any type of treatment that imparts a benefit to a patient suffering from an injury (e.g., TBI), including improvement in the condition of the patient (e.g., in one or more symptoms), delay in the progression of the condition, etc.

As used herein, the term “prevent” refers to the prophylactic treatment of a subject who is at risk of developing a condition and/or sustaining an injury (e.g., TBI) resulting in a decrease in the probability that the subject will develop conditions associated with the injury.

A “therapeutically effective amount” of a compound or a pharmaceutical composition refers to an amount effective to prevent, inhibit, or treat a particular injury and/or the symptoms thereof. For example, “therapeutically effective amount” may refer to an amount sufficient to modulate the pathology associated traumatic brain injury in a patient.

As used herein, the term “subject” refers to an animal, particularly a mammal, particularly a human.

### II. Administration

The agents of the instant invention used to treatment, inhibition, reduction, and/or prevention of the symptoms and/or pathology associated with traumatic brain injury (e.g., cognitive impairment, increased seizure rate) may be administered to a patient orally, such as a liquid consumable or pharmaceutical preparation. The term “patient” as used herein refers to human or animal subjects.

The instant invention encompasses compositions comprising BCAAs and, optionally, at least one pharmaceutically acceptable carrier or other component as described hereinabove. As used herein, “pharmaceutically acceptable carrier” includes any and all solvents, dispersion media and the like which may be appropriate for the desired route of administration of the pharmaceutical preparation. The use of such media for pharmaceutically active substances is known in the art. Except insofar as any conventional media or agent is incompatible with the molecules to be administered, its use in the pharmaceutical preparation is contemplated.

The compositions comprising at least one of the agents of the instant invention may be conveniently formulated for administration with a pharmaceutically acceptable carrier. Solubility limits of the agents within the particular pharmaceutically acceptable carrier may be easily determined by one skilled in the art.

Compositions of the instant invention may be administered orally. The pharmaceutical composition of the present invention can be prepared, for example, in liquid form (including concentrated liquid form requiring dilution (e.g., with water) prior to consumption), or can be in dried powder form (e.g., lyophilized for later reconstitution (e.g., with water). Dosage forms for oral administration include, without limitation, tablets (e.g., coated and uncoated, chewable), sustained release capsules, gelatin capsules (e.g., soft or hard), solutions, drinks, concentrates, emulsions, suspensions, syrups, elixirs, and powders/granules (e.g., reconstitutable or dispersible).

The BCAAs of the instant invention may also be administered nasally (e.g., via nasal pump, nasal spray, nasal drops, etc.). For example, a composition comprising the BCAAs can be administered as an aerosol formulation which contains the BCAAs in dissolved, suspended or emulsified form in a propellant or a mixture of solvent and propellant. The aerosolized formulation is then administered through the respiratory system or nasal passages. An aerosol formulation used for nasal administration may be an aqueous solution designed to be administered to the nasal passages in drops or sprays. The nasal solutions may also be administered without propellant—e.g., via nasal drops, spray or mist (e.g., an atomized spray). Nasal solutions are generally prepared to be similar to nasal secretions and are generally isotonic and slightly buffered to maintain a pH of about 5.5 to about 6.5, although pH values outside of this range can additionally be used (e.g., a pH of about 5 to about 8). Antimicrobial agents or preservatives can also be included in the formulation.

Pharmaceutical compositions containing agents of the present invention as the active ingredient in intimate admixture with a pharmaceutically acceptable carrier can be prepared according to conventional pharmaceutical compounding techniques. The carrier may take a wide variety of forms depending on the form of preparation desired for administration, e.g., oral.

The compounds/compositions of the instant invention may also be encapsulated within a protective material (e.g., one that is polymeric in nature or one which is lipid-based (e.g., liposomes or micelles)). The polymeric drug delivery system may be colloidal or non-colloidal in nature and may be composed of biodegradable or non-biodegradable polymeric materials. Colloidal polymeric encapsulation structures include, without limitation, microparticles, microspheres, nanoparticles, and nanospheres, block copolymer micelles, and the like. In a particular embodiment, the compounds and/or compositions of the instant invention may be formulated within a nanomaterial carrier. Suitable nanomaterial carriers are known to those skilled in the art. In a particular embodiment, the nanomaterial carrier is a nanoparticle or a nanosphere. Nanoparticles or nanospheres typically range from about 1 to 1,000 nanometers (nm), particularly from about 50 to 300 nm. The term “nanosphere” refers to a type of nanoparticle that is approximately spherical in shape. The nanosphere may have a hollow core within which one or more compounds can be placed.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art.

Dosage units may be proportionately increased or decreased based on the weight of the patient. Appropriate concentrations for alleviation of a particular pathological condition may be determined by dosage concentration curve calculations, as known in the art.

In accordance with the present invention, the appropriate dosage unit for the administration of compositions of the instant invention may be determined by evaluating the toxicity of the molecules or cells in animal models. Various concentrations of the agents in pharmaceutical preparations may be administered to mice, and the minimal and maximal dosages may be determined based on the beneficial results and side effects observed as a result of the treatment. Appropriate dosage unit may also be determined by assessing the efficacy of the treatment in combination with other standard drugs. The dosage units may be determined individually or in combination with each treatment according to the effect detected.

The compositions of the instant invention may be administered at appropriate intervals, for example, at least twice a day or more until the pathological symptoms are reduced or alleviated, after which the dosage may be reduced to a maintenance level. The appropriate interval in a particular case would normally depend on the condition of the patient.

In a particular embodiment, the composition(s) of the instant invention are administered immediately or soon after the traumatic brain injury event. For example, the pharmaceutical preparation is administered at least within a month of injury, within two weeks of injury, within about the first 7 days after injury, within about the first day after injury, within about the first hour after injury.

The compositions of the instant invention may be administered continually (e.g., every day) after the injury for at least one week, particularly at least two weeks, at least three weeks, at least four weeks or more.

In general, the compositions of the instant invention may contain other components in amounts that do not detract from the preparation of effective safe formulations. The compositions of the instant invention may further comprise at least one preservative, stabilizer, carriers, excipients, and/or antibiotic. The compositions and methods of the instant invention may also be combined with other compositions and methods for the treatment of the symptoms and/or pathology of a traumatic brain injury.

The following examples describe illustrative methods of practicing the instant invention and are not intended to limit the scope of the invention in any way.

### Example 1

Lateral fluid percussion injury (LFPI; McIntosh et al. (1989) Neurosci., 28:233-244) results in changes in network excitability in the mouse hippocampus, notably a decrease in net synaptic efficacy excitability in area CA1 and an increase in net synaptic efficacy in the dentate gyrus. Dietary supplementation with branched chain amino acids (BCAAs) leucine, isoleucine, and valine initiated 48 hours after LFPI and maintained for 5 days restores net synaptic efficacy in the mouse hippocampus and re-instates hippocampal dependent cognitive function at 7 days following LFPI (Cole et al. (2010) PNAS 107:366-371).

### Materials and Methods

### Fluid Percussion Injury (FPI):

After craniectomy, a Luer-loc needle hub (3 mm inner diameter) was secured above the skull opening. On the next day, the hub was filled with saline and connected via high-pressure tubing to the FPI device. The injury was induced by a brief pulse of saline onto the intact dura. Sham animals received all of the above except the fluid pulse.

### BCAA Diet:

Two days after FPI, animals received the BCAA diet via water bottle or by gavage feeding. Animals that received the water bottles (100 mM) were able to consume the diet ad libitum. Animals that received the gavage were fed (60 g/70 kg) every day. Animals received an LFPI and two days later BCAA diet was initiated for a duration of 2, 3, 4, 5, or 10 days. Following diet administration, animals were tested for cognitive improvement via conditioned-fear response. An additional group received the diet for 5 days and subsequently had the diet removed for 5 days prior to undergoing behavioral testing.

### Behavior:

Animals were tested for anterograde cognitive recovery using conditioned fear response (CFR) at 4, 5, 6, 7, or 12 days after injury. On day 1, animals were placed in the CFR box for a total of 3 minutes. At minute 2:32, animals received a 2-second, 1.07 mA shock and remained in the box for 30 additional seconds. The following day, animals are placed in the CFR box and observed every 5 seconds for a total of 5 minutes. A total of 60 observations were used to determine freezing percentage. Freezing percentages were calculated based on the number of times over a 5 minute period that the animal froze.

### Results

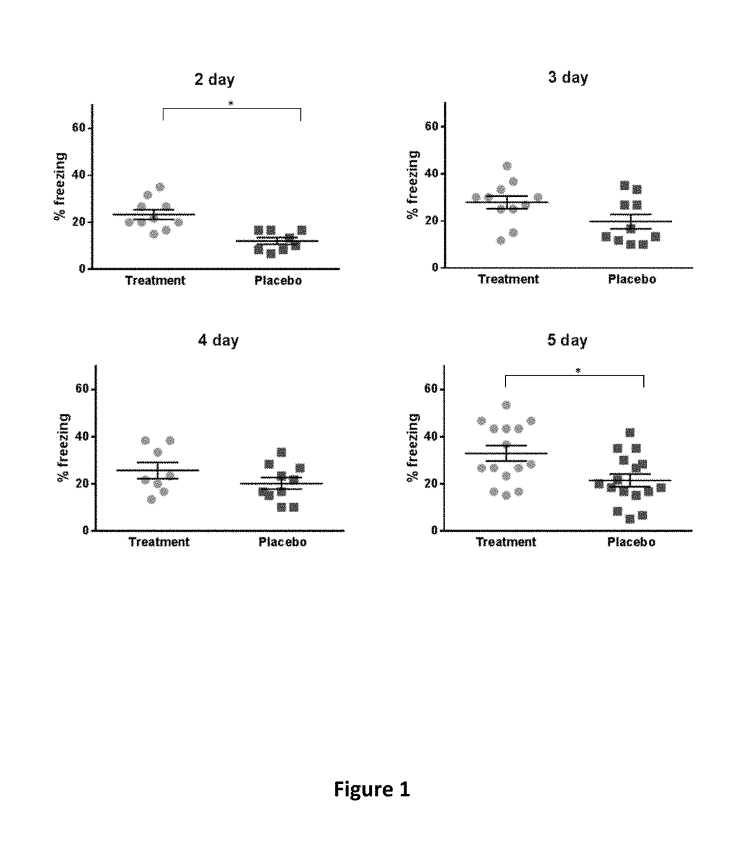

To determine the concentration and the duration of BCAA supplementation necessary to maximally enhance cognitive reinstatement, cognitive function was evaluated using conditioned fear response (CFR). Briefly, mice were administered an LFPI and placed on a 100 mM BCAA dietary supplement for 2, 3, 4, 5, or 10 days. As seen in FIG. 1, the length of time of administration correlated with an increase efficacy in CFR.

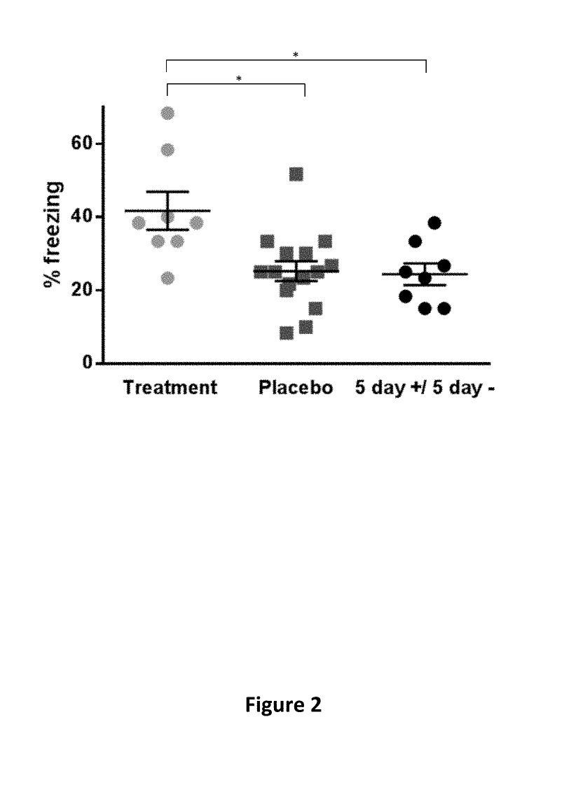

As seen in FIG. 2, the administration of BCAAs for 10 days following FPI improves performance in CFR when compared to injured mice without BCAAs. A third group of mice were placed on the diet for 5 days and then taken off the diet for 5 days. When compared to injured mice that received the diet for 10 days, the 5 day on/5 day off animals did not show significant improvements in cognition as assayed with CFR.

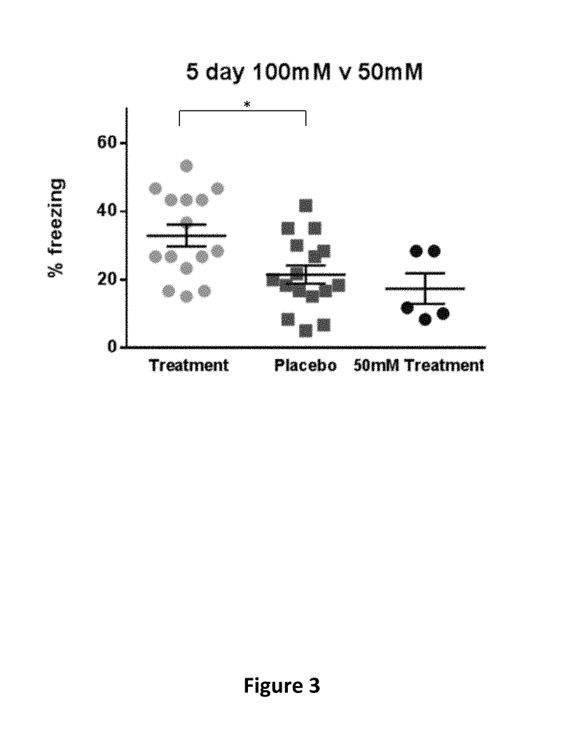

As seen in FIG. 3, decreasing the original BCAA concentration of 100 mM to a concentration of 50 mM was not efficacious in restoring cognitive ability in FPI mice when treating for 5 days.

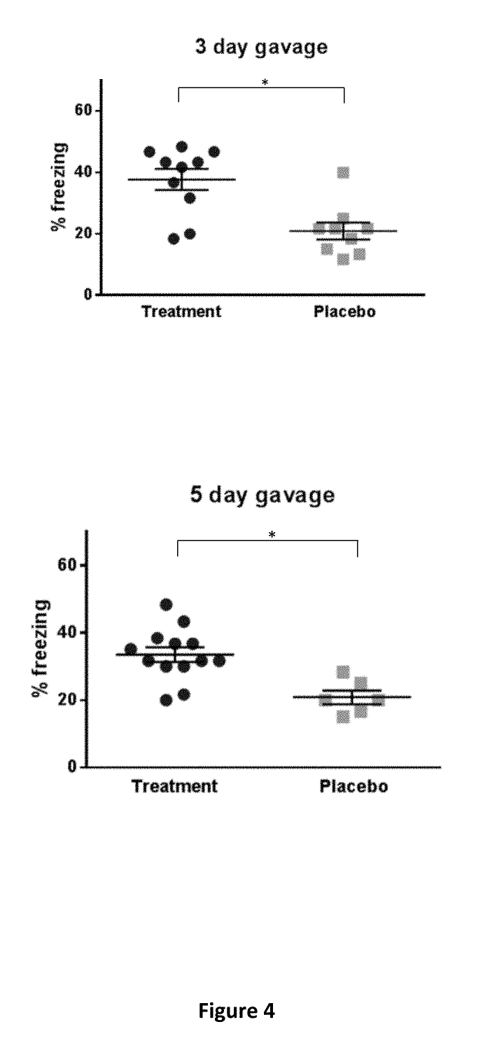

FIG. 4 shows an improved efficacy as a result of a dose specific treatment with BCAAs for 3 days and 5 days compared to injured alone. Specifically, animals received FPI and two days later received the BCAA diet via gavage feeding at a dose of 60 mg/70 kg/day. Following diet administration for 3 or 5 days, animals were tested for cognitive improvement via conditioned-fear response. Animals receiving gavage feeding showed improved performance when compared to injured alone.

These results indicate that brain injured animals need to remain on the diet to retain restorations in cognitive function. Furthermore, dietary intervention for five days is insufficient to permanently reverse alterations in glial-neuronal function caused by LFPI indicating clinical therapies for TBI may require a protracted time course.

Table 1 provides the average freezing percentage and standard deviation by condition. The data demonstrate that by 10 days animals are freezing at similar percentages as sham animals. Animals on the diet show improved performance when compared to animals from the same time point that did not undergo dietary therapy.

TABLE 1
The average incidence of freezing over a 5-minute observation
period and standard deviations.

| | With Treatment | Without Treatment |
|---|---|---|
| Sham | — | 38.81 ± 11.77 |
| 2-Day | 21.46 ± 5.38 | 11.00 ± 4.01 |
| 3-day | 25.24 ± 9.20 | 18.75 ± 8.90 |
| 4-Day | 25.63 ± 9.76 | 18.10 ± 6.83 |
| 5-Day | 32.89 ± 12.57 | 21.46 ± 10.63 |
| 10-Day | 40.67 ± 16.77 | 25.97 ± 10.60 |
| 5-Day (50 mM) | 17.33 ± 10.11 | — |

The above results show that the administration of BCAAs via water bottle or gavage feeding to FPI animals improves behavioral performance in CFR when compared to injured animals at the same time point that did not undergo BCAA dietary therapy. It is necessary for animals to continually receive the diet for at least 10 days in order to show performance similar to sham animals.

### Example 2

A fluid percussion injury assay was performed. Briefly, a craniotomy was performed on anesthetized mice using a 3 mm trephine and a Luer-Hoc needle hub attached to the skull and capped. The following day, a fluid percussion injury device was employed to deliver a pulse of saline through the hub at a pressure between 1.4-2.14 atm, producing a mild to moderate percussion injury. Sham animals received all anesthesia and surgical procedures except for the fluid pulse.

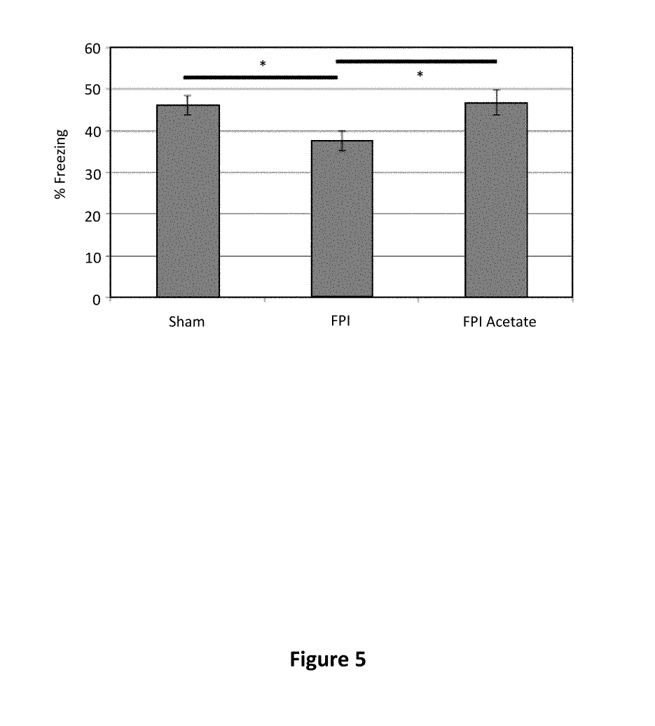

Mice were allowed to recover for one day. Mice were then treated with 5 mg/kg of glyceryl triacetate (GTA) via oral gavage for 5 days. The conditioned fear response (CFR) assay was performed on day 7. As seen in FIG. 5, mice treated with GTA after injury showed a similar fear response to sham animals and a statistically superior (P<0.05) response to those mice receiving the brain injury without treatment.

### Example 3

Recent data from the Centers for Disease Control estimates that traumatic brain injury (TBI) afflicts over 2 million people in the nation annually and is a major cause of disability in all age groups. Even mild TBI or concussion can have chronic neurological sequelae, including cognitive, motor, and sleep problems (McCrea et al. (2002) Neurosurgery 50:1032-1040; Giza et al. (2001) J. Athl. Train., 36:228-235). Sleep disorders are highly prevalent in patients with TBI (Castriotta et al. (2010) CNS Drugs 25:175-185; Castriotta et al. (2007) J. Clin. Sleep Med., 3:349-356). Sleep disturbances have been reported in up to 72% of patients with TBI (including mild TBI) up to three years post-injury (Baumann et al. (2007) Brain 130:1873-1883; Kempf et al. (2010) J. Neurol. Neurosurg. Psychiatry 81:1402-1405). TBI patients with sleep disturbances have longer inpatient hospital stays, a higher cost of rehabilitation, and a higher rate of functional disability (Makley et al. (2008) Neurorehabil. Neural Repair 22:341-347; Makley et al. (2009) Neurorehabil. Neural Repair 23:320-326). Moreover, sleep disruption is known to impair memory formation and may exacerbate cognitive deficits in TBI (McDermott et al. (2003) J. Neurosci., 23:9687-9695; Ruskin et al. (2004) Eur. J. Neurosci., 19:3121-3124). At present, there are no proven therapies to mitigate or prevent the neurocognitive and neurobehavioral consequences of TBI (Castriotta et al. (2009) J. Clin. Sleep Med., 5:137-144). Therefore, there is an imminent need to understand the neural mechanisms underlying brain injury and chronic sleep disturbances and a need for alternative therapeutic options.

Seventy-five percent of reported TBI cases are mild in nature (i.e., concussion), and sleep disturbances have been reported in 47% of 639 patients presenting to a minor head injury clinic (Haboubi et al. (2001) Disabil. Rehabil., 23:635-638). A widely-accepted, commonly used mouse model of mild TBI is the lateral fluid percussion injury (FPI) model (Dixon et al. (1987) J. Neurosurg., 67:110-119; McIntosh et al. (1989) Neuroscience 28:233-244). This experimental method provides a highly-reproducible, closed head injury that recapitulates many key features of human TBI including memory deficits, gliosis, and electrophysiological perturbation (Dixon et al. (1987) J. Neurosurg., 67:110-119; McIntosh et al. (1989) Neuroscience 28:233-244). A variety of assays have been used to demonstrate that this animal model recapitulates chronic sleep disturbances after FPI. For example, long term activity monitoring using a previously validated locomotor beam break assay was used (Pack et al. (2007) Physiol. Genomics 28:232-238). In order to further investigate changes in activity profiles, EEG/EMG recordings in freely behaving mice were performed, which allowed assessment of non-rapid-eye-movement (NREM) sleep, rapid-eye-movement (REM) sleep and wake states, and power spectral analyses.

To investigate the neural mechanisms underlying injury-induced sleep disturbances, the neuropeptide orexin was examined, which is involved in maintaining wakefulness (Chemelli et al. (1999) Cell 98:437-451). Cerebrospinal fluid (CSF) orexin levels and hypothalamic orexin neuron numbers are significantly decreased in both narcolepsy (a human sleep disorder characterized by excessive daytime sleepiness and sleep fragmentation) as well as after human TBI (Chemelli et al. (1999) Cell 98:437-451; Baumann et al. (2009) Ann. Neurol., 66:555-559; Baumann et al. (2005) Neurology 65:147-149; Krahn et al. (2002) Sleep 25:733-736). Therefore, mild TBI may cause dysfunction of the orexin system.

Injury-induced deficits in the orexin system may be reversed with dietary branched chain amino acid (BCAA) supplementation. BCAA serve as precursors for de novo glutamate and subsequent GABA synthesis in the brain. It has been shown that FPI induces a secondary cascade of injury that affects the balance of excitation to inhibition (E-I) and causes regional shifts in network excitability in the hippocampus, all of which are restored with BCAA therapy (Cole et al. (2010) Proc. Natl. Acad. Sci., 107:366-371; Witgen et al. (2005) Neuroscience 133:1-15). In addition, dietary amino acids have been shown to act directly on orexin neurons to modulate membrane excitability (Karnani et al. (2012) Neuron 72:616-629). Herein, mice were given BCAA supplementation in their drinking water and reinstated both orexin neuron activation and injury-induced sleep disturbances, indicating that BCAA restore wakefulness in part by activating orexigenic (hypocretinergic) neurons.

The data presented herein identify novel mechanisms underlying sleep disturbances in a model of mild TBI and show for the first time that BCAA intervention ameliorates injury-induced sleep disturbances, thereby identifying a therapy for the cognitive and neurobehavioral sequelae from mild TBI.

### Methods

### Animals

All experiments were performed on 5-7 week old, 20-25 g, male C57BL/J6 mice (Jackson Laboratory). The animals were housed in an insulated and soundproof recording room that was maintained at an ambient temperature of 23±1° C. with a relative humidity of 25±5% and that was on an automatically controlled 12-h light/12-h dark cycle (light on at 07:00 hours, illumination intensity≈100 lux). The animals had free access to food and water. Every effort was made to minimize the number of animals used and any pain and discomfort experienced by the subjects. Animal experiments were performed in accordance with the guidelines published in the National Institutes of Health Guide for the Care and Use of Laboratory Animals and were approved by the University of Pennsylvania and Children's Hospital of Philadelphia Animal Care and Use Committee in accordance with international guidelines on the ethical use of animals.

### Mouse Fluid Percussion Brain Injury

Mice were divided into two groups: TBI (surgery and fluid percussion injury) and Sham. The fluid percussion brain injury (FPI) protocol was carried out over 2 days as previously described (McIntosh et al. (1989) Neuroscience 28:233-244; Cole et al. (2010) Proc. Natl. Acad. Sci., 107:366-371). On the first day, the animal was anesthetized using a combination of ketamine (100 mg/kg) and xylazine (10 mg/kg) and placed in a mouse stereotactic frame (Stoelting). The scalp was incised and reflected. The following was conducted under 0.7-3.5× magnification: A craniectomy was performed with a trephine (3-mm outer diameter) over the right parietal area between bregma and lambda, just medial to the sagittal suture and lateral to the lateral cranial ridge. The dura remained intact throughout the craniotomy procedure. A rigid Luer-lock needle hub (3-mm inside diameter) was secured to the skull over the opening with Loctite® adhesive and subsequently cyanoacrylate plus dental acrylic. The skull sutures were sealed with the cyanoacrylate during this process to ensure that the fluid bolus from the injury remained within the cranial cavity. The Luer-lock needle hub was filled with isotonic sterile saline and the hub was capped. The mouse was then placed on a heating pad and returned to the home cage once ambulatory. On the second day, the animal was briefly placed under isoflurane anesthesia (500 mL/min) via nose cone, and respiration was visually monitored. When the animal was breathing once per 2 seconds, the nose cone was removed, the cap over the hub removed, and dural integrity visually confirmed. The hub was topped off with isotonic sterile saline, and a 32-cm section of high-pressure tubing extending from the FPI device attached to the Luer-lock fitting of the hub (Department of Biomedical Engineering, Virginia Commonwealth University, Richmond, Va.). The animal was then placed on its left side and observed. Once normal breathing resumed and just as the animal regained its toe pinch withdrawal reflex, a 20-ms pulse of saline onto the dura was delivered. A pressure gauge attached to an oscilloscope was used to ensure delivered pressures between 1.4 and 2.1 atmospheres, which have been previously shown to generate a mild brain injury. Immediately after injury, the hub was removed from the skull and the animal was placed in a supine position. The animal was then reanesthetized with isoflurane for scalp closure. Sham animals received all of the above, with the exception of the fluid pulse. The animal was returned to a heating pad until ambulatory and then returned to the home cage.

### Assessment of Activity and Inactivity: Infrared Beam Breaks

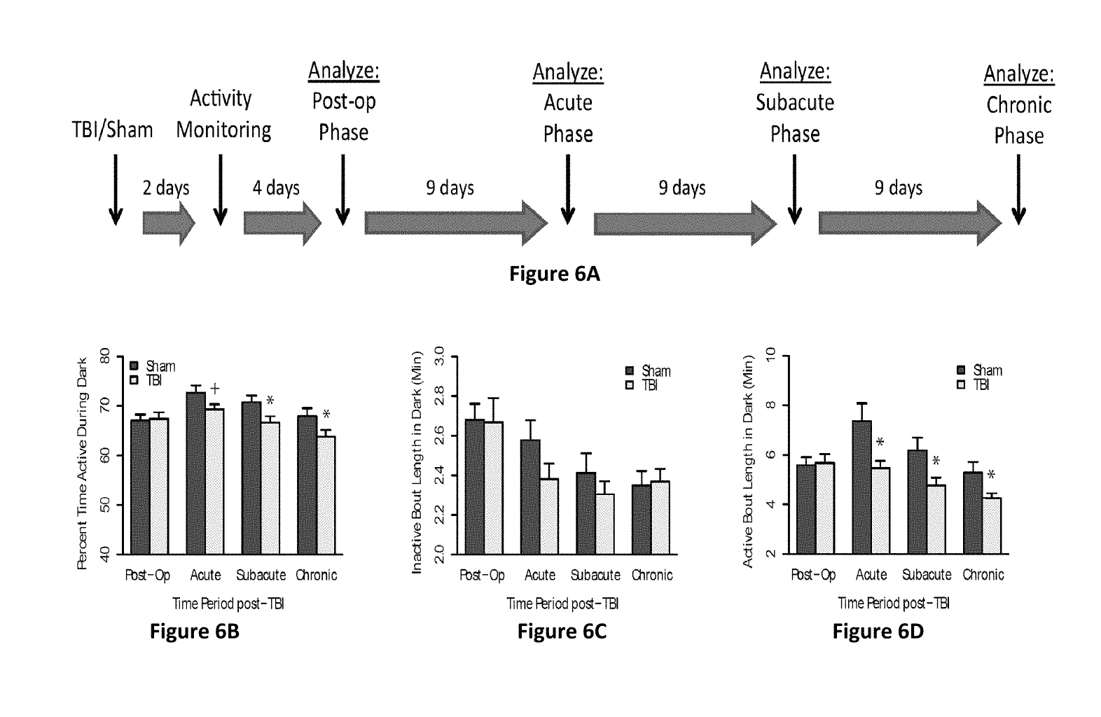

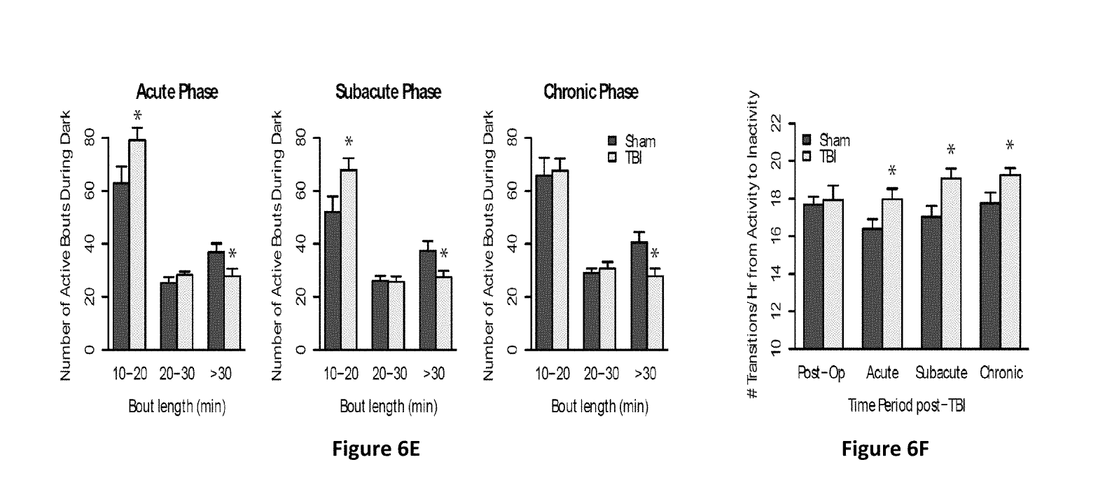

The activity monitoring timeline is detailed in FIG. 6A. Activity/inactivity was determined using the Accuscan monitoring system (Omnitech Electronics, Inc.) and Fusion 4.0 software collection system. Following FPI or sham surgery, mice were individually housed in their home cages within the Accuscan monitoring system for 30 consecutive days (n=18 FPI, n=12 sham). The Accuscan system consists of infrared beam that are 1 inch apart on the horizontal plane providing a high-resolution grid covering cage bottom. Data acquisition software provides counts of beam breaks by the mouse in 10 second epochs. The mouse was considered inactive if there were no beam breaks in four consecutive 10 second epochs, in accordance with an algorithm developed to estimate sleep and wakefulness (Pack et al. (2007) Physiol. Genomics 28:232-238).

### EEG/EMG Assessment of Sleep and Wakefulness

To assess sleep and wakefulness, mice were surgically implanted with EEG/EMG electrodes as previously described, with slight modifications for the craniotomy from FPI (Pack et al. (2007) Physiol. Genomics 28:232-238). Briefly, animals were anesthetized by injection of ketamine (100 mg/kg) and xylazine (10 mg/kg). The skull was exposed and 3 small holes prepared for placement of 3 silver ball EEG electrodes, two frontal and one left parietal (anteroposterior, +1.0 mm; mediolateral, +1.5 mm from bregma, and anteroposterior, −2.0 mm; mediolateral −2.0 mm) according to the atlas of Franklin and Paxinos (Paxinos, G. (2004). The mouse brain in stereotaxic coordinates: Gulf Professional Publishing). Two insulated stainless steel EMG electrodes bared at the tips were buried on the surface of dorsal neck muscles. All leads from the electrodes were connected to a plastic socket connector (Plastics One), which was fixed to the skull with dental acrylate. Following surgery, animals were allowed to recover for 5 days before any studies were performed. EEG and EMG signals were amplified using the Neurodata amplifier system (model M15, Astro-Med Inc.). Signals were amplified (20,000×) and conditioned with neuroamplifiers/filters (model 15A94, Grass). Settings for EEG signals were a low cut frequency (−6 dB) of 0.1 Hz and a high cut frequency (−6 dB) of 100 Hz. Samples were digitized at 256 Hz samples/second/channel. All data were acquired using Grass Gamma software (Natus).

### Branched Chain Amino Acid (BCAA) Administration

Two days after FPI procedure, a subset of mice was randomly assigned to receive either BCAA supplemented water, or untreated tap water (control). BCAA supplementation consisted of a combination of L-Leucine, L-isoleucine, and L-Valine at 100 mM each (obtained individually from Sigma-Aldrich) (Cole et al. (2010) Proc. Natl. Acad. Sci., 107:366-371). The amount of drinking water remaining in the bottle was measured each day, and fresh BCAA or control water replaced each week. Mice drank on average 3-5 mL of solution per day and it has been shown that BCAA do not affect body weight (Cole et al. (2010) Proc. Natl. Acad. Sci., 107:366-371).

### Recording Timeline and Behavior Testing

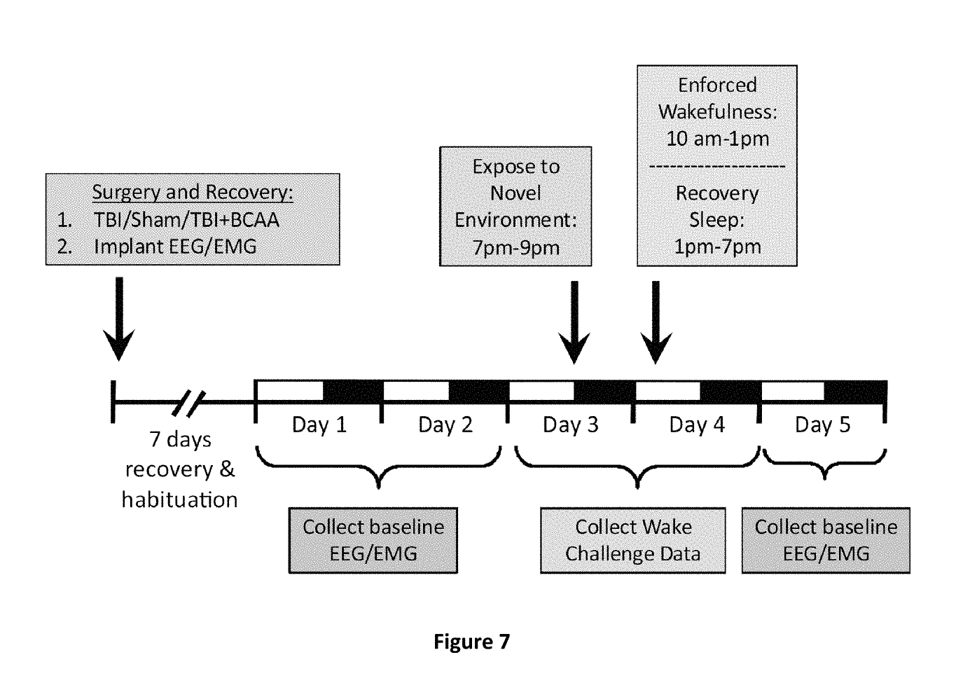

The EEG/EMG recording timeline is shown in FIG. 7. Mice were connected to lightweight recording cables in individual cages (n=7 Sham, n=6 TBI, n=6 TBI+BCAA). Sleep recordings were initiated after 24 hours of acclimation to the cables. Ability to move freely within the entire cage and stand on hind limbs to explore the top of the cage was confirmed in all mice studied. Baseline sleep was recorded on the first, second and fifth days to ensure stable sleep/wake activity across days. At the start of the dark phase (7:00 pm) on recording day 3, mice were exposed to a novel environment without direct handling by adding new bedding and a nestlet to their home cages. EEG/EMG signals were recorded and analyzed from 7:00 to 9:00 pm for response to a novel environment. On recording day 4, mice were sleep deprived using gentle handling for 3 hours, from 10:00 am to 1:00 pm, which is a time of heightened sleep. Gentle handling was accomplished by providing the mice with novel materials (bedding, nestlets, pieces of paper towels, aluminum foil, and saran wrap) and occasionally stroking the mice with a soft paintbrush as described (Naidoo et al. (2011) Aging Cell, 10:640-649). During this enforced wakefulness, wake was electrographically confirmed during the entire 3 hour period. Subsequently, recovery sleep for the next three hours was recorded and analyzed for wake probability and delta power as described below in Data Analysis.

### Data Analysis: Baseline EEG/EMG Scoring

Polygraphic records were scored offline by an experienced scorer for non-rapid-eye movement (NREM) and rapid-eye-movement (REM) sleep and wakefulness (W) in 4 second epochs across a 24 hour baseline (12 hour light-dark cycle from 7:00 am to 7:00 pm). Data collected using the Grass Gamma software were converted to European Data Format and automatically scored using the SleepSign analysis package according to standard criteria (Kissei Comtec).

Automatically scored epochs were then visually inspected for artifact and manually removed (<0.5% of all epochs). As a final step, defined sleep-wake stages were examined and corrected if necessary. Over a baseline period of 24 hours consisting of exactly 21,600 epochs per mouse, total minutes of Wake, NREM, and REM sleep, the average length of each sleep and wake bout, and the characteristics of the transitions within and between sleep and wake states were quantified.

### Data Analysis: Baseline Power Spectral Analysis

Scored polygraphic signals underwent Fast Fourier Transform (FFT) using SleepSign for power spectral analysis of each 0.25 Hz frequency bin from 0 to 128 Hz in total for each sleep stage (NR, R, and W). The data were then aggregated using R for power density within particular EEG frequency bands: Delta (1-4 Hz), theta (5-8 Hz), alpha (9-13 Hz), and beta (14-30 Hz), total gamma (31-128 Hz), slow gamma (35-55 Hz) and total power (1-128 Hz). The EEG power density for each epoch was normalized by the total power averaged from all epochs and summed within each frequency bin for each sleep stage. Epochs containing artifacts were eliminated from power spectral analysis. Theta peak frequency was calculated by identifying the frequency between 5 and 8 Hz which had the greatest power density for each individual mouse, and then averaging this value within groups.

### Data Analysis: Novel Environment

Polygraphic records over the analysis period for Novel Environment were extracted from 7:00 to 9:00 pm on recording day 3, and scored in the same manner as detailed above. The latency to the first sleep period, defined as 3 consecutive NREM or REM epochs in any combination, was quantified for each mouse. The chronological plot of percentage wakefulness was calculated by tabulating the total number of wake epochs within consecutive 5 minute bins and expressing this as a percentage of total epochs, plotted over 120 minutes.

### Data Analysis: Recovery Sleep

Polygraphic signals were extracted from the Recovery Sleep period occurring immediately after a 3 hour period of enforced wakefulness, from 1:00 pm to 7:00 pm on recording day 4. The epochs were scored as above and analyzed for total NREM time and average NREM bout length. Delta power was calculated by quantifying the average NREM power for each hour immediately following sleep deprivation from 1:00 pm to 7:00 pm, and then normalized by dividing by the baseline NREM delta power for each individual mouse. The baseline NREM delta power was calculated by averaging values over the last four hours of Lights On (3:00 pm to 7:00 pm) during baseline day 3, where values are presumably at their zenith (Franken et al. (2001) J. Neurosci., 21:2610-2621). The chronological plot of percentage wakefulness was calculated by tabulating the total number of wake epochs within consecutive 5 minute bins and expressing this as a percentage of total epochs, plotted over 3 hours.

### Immunohistochemistry

Immunohistochemistry was used to characterize the waking c-fos response in orexin neurons, an immediate early gene marker of recent neural activation. Mice were perfused with 4% paraformaldehyde, and brains were post-fixed then cryopreserved in sucrose. Cryostat sections were collected at 40 um thickness in a 1:6 series. Sections containing the lateral hypothalamus underwent double-labeling for orexin and c-Fos as described (Naidoo et al. (2011) Aging Cell 10:640-649). Briefly, sections were incubated in goat polyclonal anti-Orexin-A antibody (Santa Cruz) at 1:1000 concentration and rabbit polyclonal anti-c-Fos antibody (Calbiochem) at 1:6000 concentration. Orexin-A was visualized with an anti-goat Alexa Fluor® 594 (red) secondary antibody at 1:200, and c-Fos was visualized with an anti-rabbit Alexa Fluor® 488 (green) secondary antibody at 1:200 (Molecular Probes). For Vesicular Glutamate Transporter 1 (VGLUT1) and Orexin-A double-labeling, the protocol was the same with the exception of using rat polyclonal anti-VGLUT1 antibody (Synaptic Systems) at 1:2000 concentration. Sections were mounted onto microscope slides and analyzed for cell counting using an epifluorescent microscope (Leica) at 10× and 40× magnification as previously described (Naidoo et al. (2011) Aging Cell 10:640-649).

### Cell Counting

All immunopositive orexin-A neurons with visible nuclei in four rostral-caudal sections across bilateral nuclei for each mouse were analyzed (n=5 mice per group). Cells were scored as c-Fos positive if c-Fos labeling in the nucleus was more intense than background using Image J analysis software (NIH) as previously described (Naidoo et al. (2011) Aging Cell 10:640-649). Two scorers, one of whom was blinded to conditions, independently counted orexin neuron numbers and c-Fos labeling with >90% agreement. For each mouse, greater than 150 neurons per region were examined.

### Statistical Procedures

Statistical calculations and analyses were performed using R (Version 2.15.2, The R Foundation of Statistical Computing) (Team, R. C. (2012) R: A language and environment for statistical computing. Vienna, Austria: R Foundation for Statistical Computing). Where appropriate, all data were analyzed using either two-tailed Student's t-tests or a one-way ANOVA followed by Dunnett's post-hoc tests if the F values reached statistical significance. Statistical significance was defined at the p<0.05 confidence level when comparing different treatment groups. All data are presented as group means±SEM.

### Results

### Activity Monitoring: Decreased and Fragmented Activity

A widely-accepted, commonly used mouse model of mild TBI is the lateral fluid percussion injury (FPI) model. This experimental method provides a highly-reproducible, closed head injury that recapitulates many key features of human TBI including memory deficits, gliosis, and electrophysiological perturbation (Dixon et al. (1987) J. Neurosurg., 67:110; McIntosh et al. (1989) Neuroscience 28:233).

Mice randomized to either FPI or Sham surgery underwent extensive locomotor activity monitoring from 3 to 34 days. The experimental timeline denoting the pre-determined time blocks is shown in FIG. 6A, and consists of the immediate post-operative period (days 0-4), acute (days 5-14), subacute (days 15-24) and chronic (days 25-34) periods following injury or sham surgery. Activity patterns were analyzed for each of the time blocks. A previously established algorithm was applied in which 40 seconds of continuous inactivity was shown to be highly predictive of sleep (Pack et al. (2007) Physiol. Genomics 28:232-238). Therefore, an inactive bout was counted only when mice remained still for greater than 40 continuous seconds.

During the dark phase (7:00 pm to 7:00 am), when mice are typically more active, injured mice were significantly less active compared to Sham controls during the subacute and chronic phases (FIG. 6B). The average length of time spent continuously inactive, or “Inactive Bout Length,” did not significantly differ between groups (FIG. 6C). However, the average length of time spent continuously active, or “Active Bout Length,” was significantly shorter in TBI mice compared to controls (FIG. 6D). This indicates that mild TBI decreases total activity by causing shorter bouts of continuous activity.

TBI and Sham mice did not significantly differ in total amount of activity or in average active/inactive bout lengths during the light phase (7:00 am to 7:00 pm), when mice are typically less active.

In order to further dissect the nature of the shortened activity bouts after TBI, the frequency of bouts of varying duration, ranging from 10-20 minutes, 20-30 minutes, and greater than 30 minutes in length were examined during the dark phase. While the vast majority of bout durations are less than 10 minutes in length for both groups of mice, TBI mice have significantly more bouts in the 10-20 minute range and significantly fewer bouts greater than 30 minutes in length compared to Sham controls, throughout the acute, subacute and chronic time blocks (FIG. 6E).

TBI and Sham mice did not show the same magnitude of activity differences during the light phase, compared to the dark phase, but the pattern of decreased and shortened activity bouts was still evident in brain injured mice.

The total number of transitions between continuous Active and Inactive Bouts was significantly increased after TBI across the three time blocks (FIG. 6F). This indicates that there is significant fragmentation of activity bouts in injured mice.

To investigate whether diurnal rhythms were affected in TBI mice, actograms were plotted over the entirety of the 30 day activity monitoring period. Gross diurnal activity rhythms were intact in both Sham and TBI mice, consistent with the phenotype of an intact circadian clock. This indicates that TBI affects activity mechanisms downstream of the clock.

Taken together, the locomotor activity monitoring data demonstrate that a single episode of mild injury causes persistent alterations in activity lasting at least 30 days. Given that locomotor activity is highly predictive of behavioral state, this data indicates there are injury-induced alterations in sleep and wakefulness. In particular, brain injured mice show greater differences during the dark phase, when mice are typically more awake. Therefore, it was hypothesized that there would be specific deficits in the ability to sustain wakefulness.

### Baseline EEG/EMG Recording: Inability to Sustain Wakefulness, Improved by BCAA Intervention

To investigate the behavioral mechanisms underlying injury-induced decreases in activity, mice were implanted with chronic indwelling EEG/EMG electrodes. Behavioral states were analyzed over a five day period following an initial 7 day recovery period from surgery (FIG. 7). Because FPI causes changes in brain network excitability which are normalized by administration of dietary branched chain amino acids (BCAA), EEG/EMG recordings were performed on a separate cohort of injured mice on BCAA therapy. It was hypothesized that deficits in sleep-wake network excitability would be ameliorated by this dietary supplement in the drinking water.

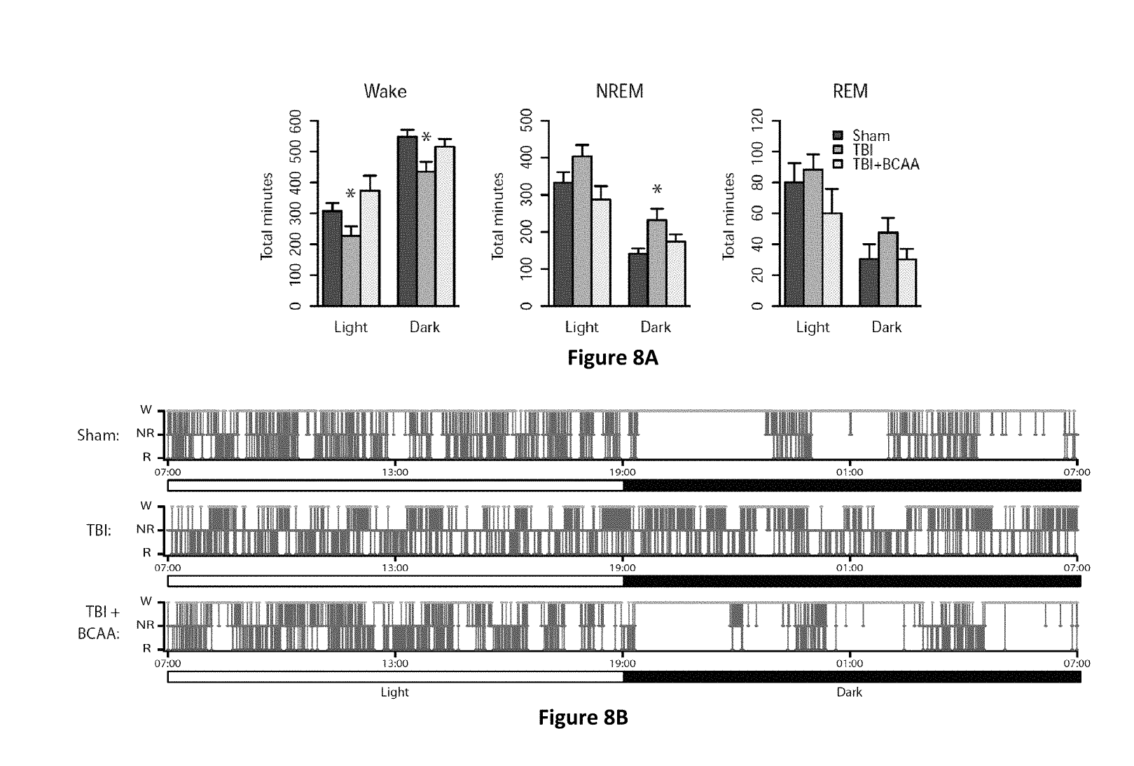

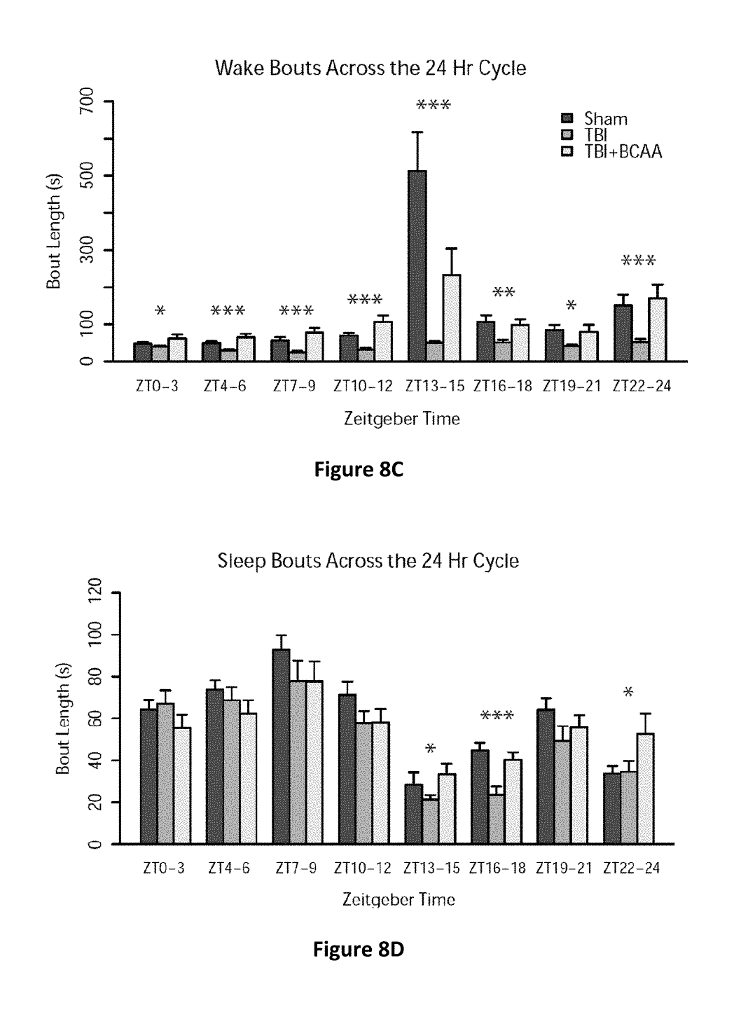

Baseline recordings consisted of 24 hour periods from 7:00 am to 7:00 am. During baseline conditions, TBI mice spend significantly less time awake over both the light and dark phases, and more time in NREM sleep during the dark phase compared to Sham controls (FIG. 8A). TBI+BCAA mice show a partial reversal of changes in wake and NREM states. These results indicate that the decrease in activity measured using locomotor monitoring is explained by reduced wakefulness and increased NREM sleep.

Hypnograms were calculated for each animal by plotting wake, NREM and REM stages consecutively epoch-by-epoch over a 24 hour period, beginning with Lights On at 7:00 am. Naïve adult C57Bl6 wildtype mice typically show long bouts of wakefulness during the dark phase, particularly at the start of lights off, whereas orexin-deficient mice lack long wake bouts (Sakurai, T. (2007) Nat. Rev. Neurosci., 8:171-181). Sample hypnograms from Sham, TBI and TBI+BCAA mice are shown in FIG. 8B. Sham mice maintain long wake bouts at 7:00 pm Lights Off, while TBI mice continue to have fragmented wake bouts. BCAA intervention reinstates prolonged wake episodes.

Average continuous wake bout lengths were quantified over the 24 hour circadian cycle, subdivided into 3 hour bins beginning with Lights On at 7:00 am (or Zeitgeber Time (ZT) 0). TBI mice showed significantly shorter wake bouts in both the light and dark phases compared to Sham mice (FIG. 8C). This is particularly marked in the early part of the lights off period. Injured mice are unable to achieve long bouts of wakefulness compared to Sham control mice. BCAA therapy significantly lengthens wake bouts throughout the 24 hour cycle, thereby improving normal maintenance of wakefulness, but does not completely restore the ability to sustain wakefulness to control levels (FIG. 8C).

Average continuous sleep bout lengths were also quantified over the 24 hour circadian cycle, subdivided into 3 hour bins beginning as above. TBI mice have significantly shorter continuous sleep bouts during the dark phase compared to Sham mice, and this is partially restored with BCAA intervention, particularly during ZT 16-18, or 10:00 pm to 1:00 am (FIG. 8D).

Overall, these data indicate that TBI impairs the ability to sustain wakefulness and disrupts the normal diurnal fluctuation seen in wakefulness. The effect is most marked in the early part of the lights off period. BCAA therapy improves the ability to stay awake.

### Baseline EEG/EMG Recording: Behavioral State Fragmentation, Rescued by BCAA Therapy

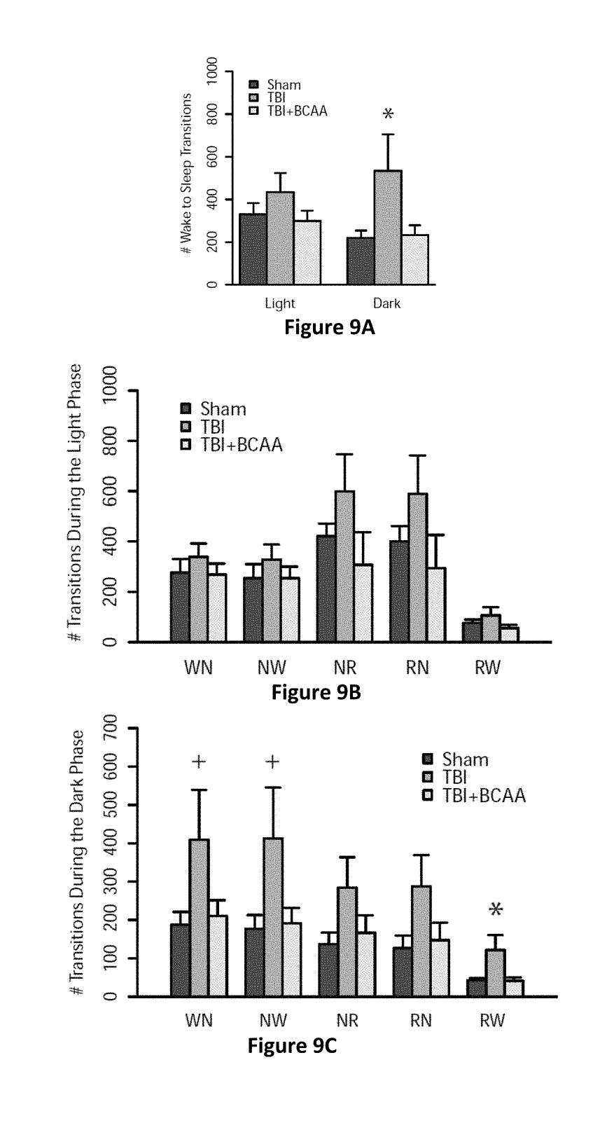

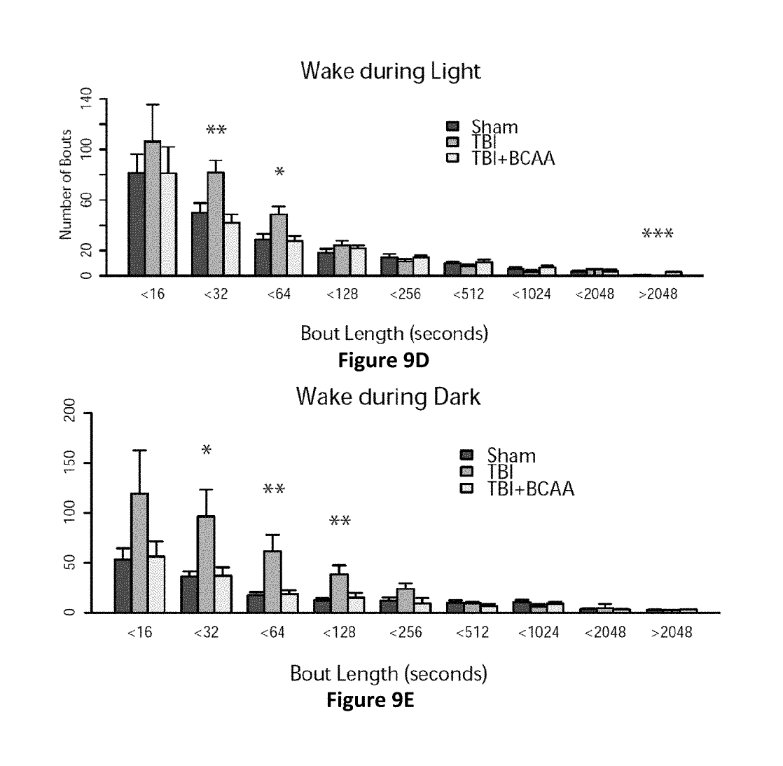

To investigate the transitions between behavioral states in brain injury, the number of sleep and wake bout switches over the light and dark phases were quantified. During the dark phase, the total number of sleep-to-wake transitions (sleep=NREM and REM) was significantly increased in TBI mice compared to Sham mice, and BCAA intervention after TBI decreased the number of transitions back to Sham control levels (FIG. 9A).

Transition types were categorized into possible combinations between Wake, NREM and REM stages and sub-divided by light and dark phases. TBI mice have more Wake to NREM (WN), NREM to Wake (NW), and REM to Wake (RW) transitions in comparison to Sham controls as well as in comparison to TBI with BCAA intervention during the dark phase (FIG. 9C). There were no significant differences between groups for transition subcategories during the light phase, when mice typically spend more time sleeping (FIG. 9B). This indicates that the transitions that specifically involve the wake state (i.e., WN, NW and RW) are most susceptible to injury.

In order to further characterize the nature of wake transitions, a histogram of varying wake bout durations was plotted (FIGS. 9D and 9E). During both the light and dark phases, TBI mice had significantly more bouts of shorter duration (in particular, less than 128 seconds in length) compared to Sham mice. BCAA therapy completely rescued this left shift towards shorter bout durations in the histogram.

Bouts were also analyzed in NREM and REM sleep during the light and dark phases. While the most pronounced group differences are during the wake state, there is a similar, consistent pattern of shorter bouts after injury during NREM and REM states, indicating some degree of sleep fragmentation.

Taken together, these data highlight the severity of fragmentation during wakefulness, and also to a lesser degree also during sleep, induced by mild TBI. Behavioral state fragmentation and excessive daytime sleepiness are phenomena frequently cited in sleep disorders such as narcolepsy and post-traumatic hypersomnia (Guilleminault et al. (2000) Neurology 54:653-659; Rao et al. (2008) Brain Inj., 22:381-386; Verma et al. (2007) J. Clin. Sleep Med., 3:357-362; Baumann et al. (2007) Brain 130:1873-1883).

### Baseline EEG/EMG Sleep-Wake Recording: Power Spectral Analysis

Power spectral analysis is a widely accepted method used for quantification of EEG signals. The power spectral density reflects the distribution of signal power (calculated by fast Fourier Transform of the polygraphic signal) plotted over specific frequency bins.

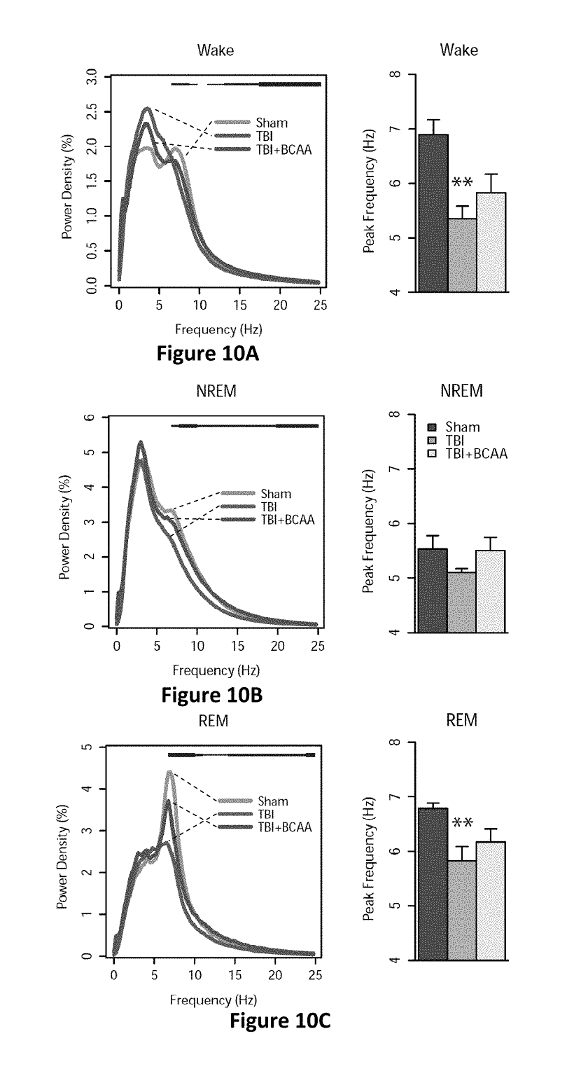

Baseline polygraphic signals were analyzed for power spectra in various frequency bands for the different conditions (FIG. 10). Wake spectra for TBI mice were significantly lower at the theta frequency range compared to Sham control mice (FIG. 10A). This was also found in NREM and REM spectra for TBI mice (FIGS. 10B and 10C). Theta power was restored by BCAA for spectra in the NREM state. Power spectra were further categorized by sleep stage and light/dark phases, which highlight group differences in particular for wake spectra during the dark and NREM/REM spectra during the light.

Because robust group differences in theta power were found, this effect was further evaluated by calculating the theta peak frequency for each behavioral state. Theta peak frequency was derived as the frequency value between 5 and 8 Hz with the maximum power density. In both wake and REM states, theta peak frequency for TBI mice was significantly slower compared to Sham control mice. BCAA therapy increased theta peak frequency, albeit not to control levels (FIGS. 10A and 10C).

Power density for specific frequency bands was calculated during each behavioral state. Group differences were noted in alpha, beta and gamma power densities, with injured mice showing a decrease in all three compared to Sham mice, with partial improvement in some bands with BCAA therapy.

Taken together, the power spectral analyses demonstrate that mild TBI produces persistent alterations in EEG rhythms. In particular, injury reduces theta power and shifts the theta peak to slower frequencies. This phenotype is ameliorated with BCAA intervention.

### EEG/EMG Recording During a Novel Environment Challenge

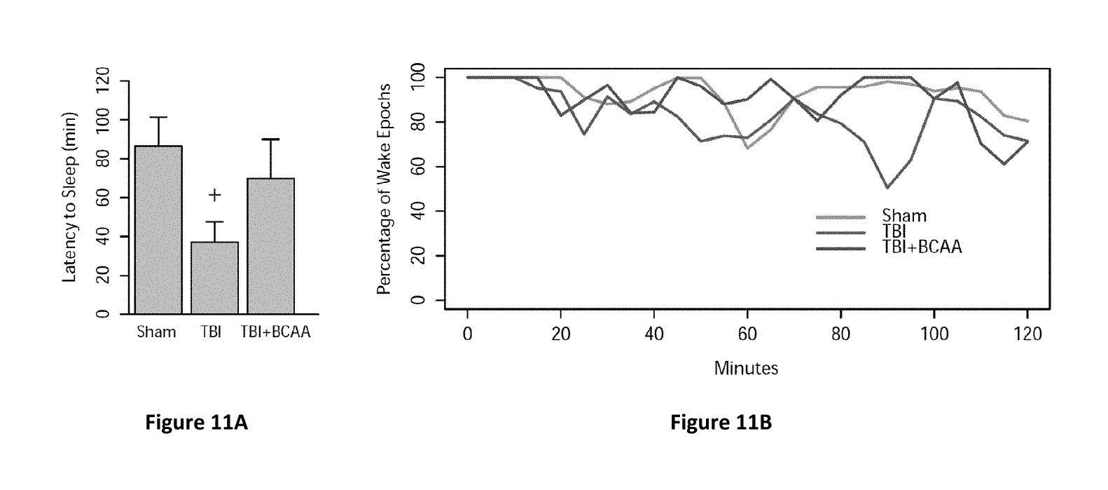

Next, it was determined whether injury affects behavioral state in situations that challenged the arousal system. Mice were exposed to a novel environment at the start of the dark phase. The latency to first sleep episode was calculated for the two hour behavioral test (FIG. 11A). TBI mice had a shorter latency to sleep compared to Sham mice, and this shorter latency was partially restored by BCAA intervention (F=2.805, p=0.0923, Sham v. TBI p=0.05; one-way ANOVA with Dunnett's post-hoc test). Next, a chronological plot of wakefulness was calculated by tallying the total number of wake epochs per 5 minute bins. This plot showed that TBI mice had consistently lower percentage of wake epochs throughout the two hour test, with increasing propensity to sleep as the test went on (FIG. 11B). Sham control and BCAA therapy mice were indistinguishable, indicating that BCAA restore normal arousal and wakefulness during exposure to novelty.

EEG/EMG Recording after Mild Sleep Deprivation

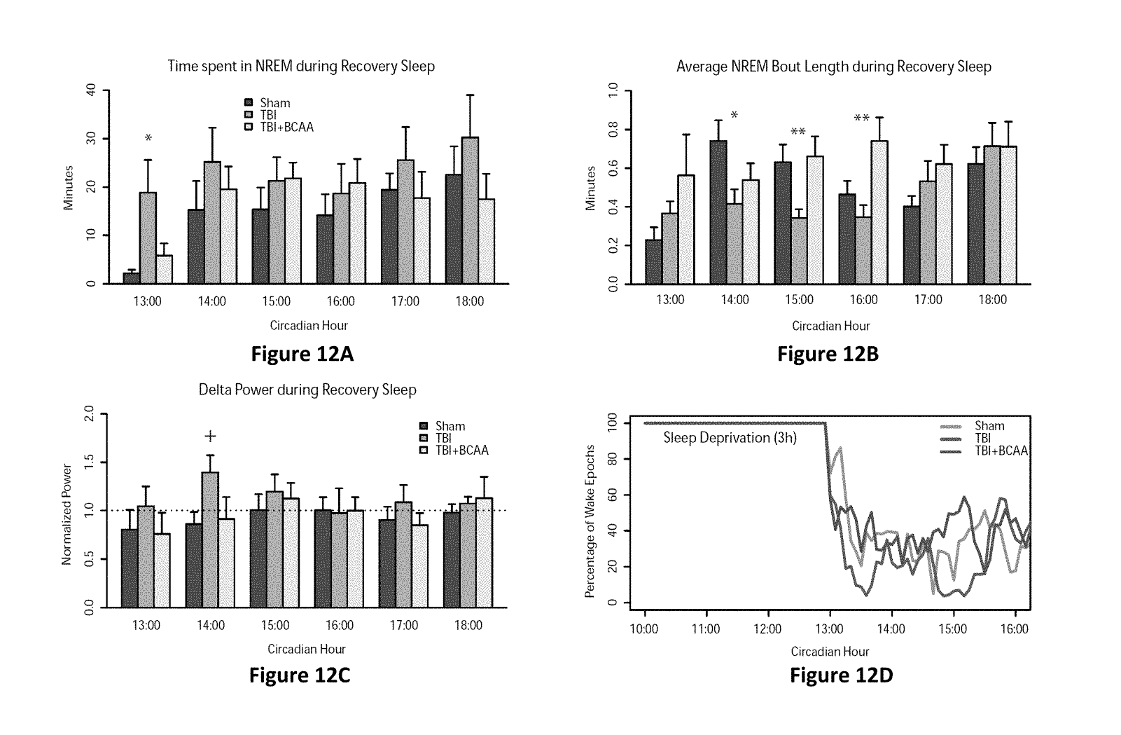

The second wake challenge consisted of examining EEG response to a 3 hour period of enforced wakefulness using sleep deprivation by gentle handling. The wake challenge was performed from 10:00 am to 1:00 pm, which is a period of high sleep pressure in mice. The 3 hour period was chosen as an abbreviated length of time during which naïve mice typically do not show much sleep rebound, so as to maximize effect size (Franken et al. (1991) Neurosci. Lett., 130:141-144). Data from the following six hours from 1:00 to 7:00 pm, or “Recovery Sleep,” were analyzed for NREM time, NREM bout length, NREM delta power and chronological percentage of wakefulness (FIG. 12).

TBI mice spent significantly more time in NREM sleep in the first hour immediately after sleep deprivation compared to Sham controls and mice receiving BCAA therapy (FIG. 12A). Interestingly, despite having more NREM sleep, TBI mice had shorter NREM bout lengths (FIG. 12B). Delta power during NREM sleep is widely accepted to be an accurate proxy for sleep pressure after a period of sleep deprivation (Franken et al. (1991) Neurosci. Lett., 130:141-144; Franken et al. (1991) Am. J. Physiol., 261:R198-208). TBI mice showed more delta power in the second hour during Recovery Sleep compared to Sham and TBI+BCAA mice (FIG. 12C). The chronological plot of wakefulness was low in all three groups, but appeared lowest in the TBI group compared to Sham and TBI+BCAA mice, particularly within the first two hours (FIG. 12D).

Taken together, these data indicate that mild brain injury imparts an increased pressure to sleep after situations of enforced arousal. However, despite increased sleep pressure, TBI mice are still unable to sustain long NREM sleep bouts. BCAA intervention improves and prolongs wakefulness after mild sleep deprivation.

Orexin Neuron Activation is Decreased after TBI and Restored by BCAA Intervention

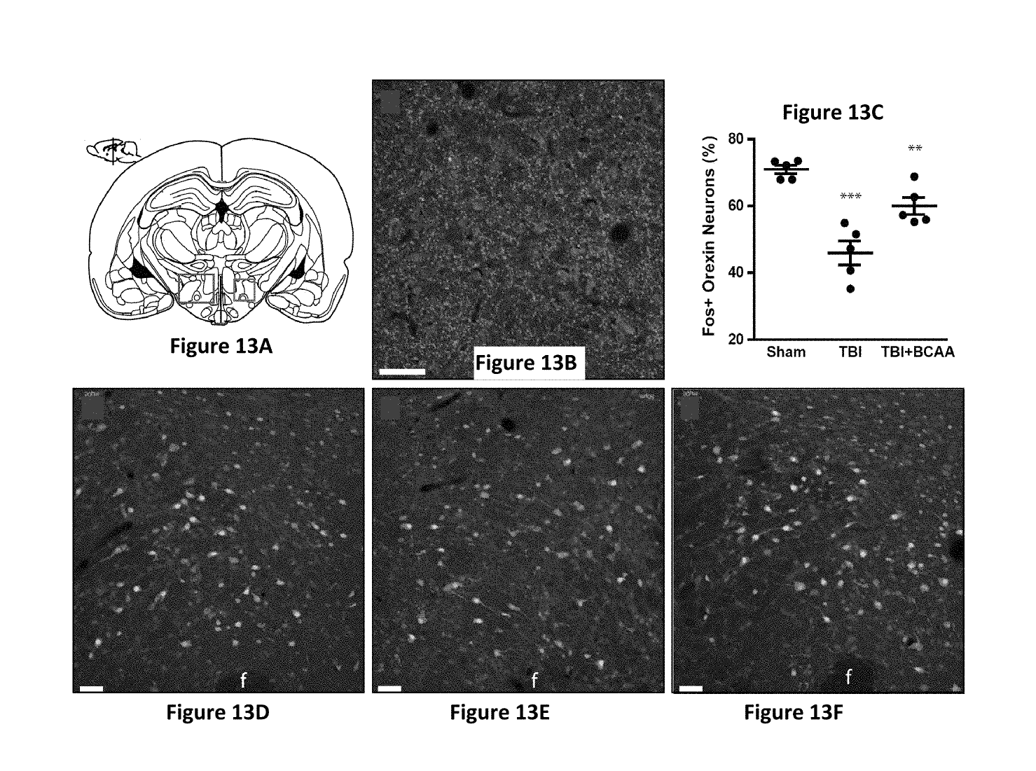

The phenotype of daytime sleepiness and state instability resembles the phenotype caused by orexin dysfunction (Sakurai, T. (2007) Nat. Rev. Neurosci., 8:171-181). Accordingly, orexin dysfunction was potentially contributing to the phenotype of injury-induced sleep disturbances. To test this hypothesis, orexin neuron activation was examined in response to a 3 hours period of enforced wakefulness from 10:00 am to 1:00 pm—the same paradigm used in the wake challenge test described above. Mice were 4 weeks post-TBI or Sham surgery, and a third group of mice received BCAA dietary supplementation for 4 weeks prior to sacrifice. Neural activation was measured by the presence of the immediate early marker c-Fos protein (Morgan et al. (1991) Annu. Rev. Neurosci., 14:421-451). Compared to Sham and TBI+BCAA groups, TBI mice had significantly fewer activated orexin neurons (FIG. 13C; F=22.47, p=0.0001; Sham v. TBI p=0.001, TBI v. TBI+BCAA p=0.01; one-way ANOVA with Dunnett's post-hoc test). Total orexin neuron numbers were not significantly different between groups, indicating that injury affects the physiological balance, and not gross cell loss per se, of this sleep-wake circuit.

Sleep disturbances, including excessive daytime sleepiness and sleep maintenance insomnia, have been reported as one prominent and chronic disabling consequence of mild traumatic brain injury (TBI). Early intervention of sleep problems would not only improve quality of life, but would also facilitate cognitive and neurobehavioral recovery after brain injury. The data presented herein are the first to establish a mouse model of chronic sleep/wake disturbances after mild TBI, namely, a persistent inability to maintain wakefulness. Herein, the locomotor activity and EEG-based behavioral states after a single mild non-penetrating head injury in mice are extensively characterized. It is then shown that orexin neuron activation is decreased in response to a wake stimulus, indicating alterations in orexin network excitability, similar to what has been described for hippocampal networks after injury (Cole et al. (2010) Proc. Natl. Acad. Sci., 107:366-371).

Given that the data show injury-induced deficits in orexin neuron activation, an intervention which restores orexin network excitability, thereby restoring wakefulness, would be effective. Indeed, dietary branched chain amino acids (BCAA) intervention rescued most sleep/wake disturbances, including increasing wake time, consolidating sleep and wake bouts, and raising arousal level to that seen in control mice during situations of heightened wakefulness. BCAA therapy achieves this effect by restoring EEG power spectral peaks, in particular theta power, and reinstating activation of orexin neurons during wakefulness.

It was found that a single mild brain injury causes persistent alterations in activity patterns, notably for over 4 weeks post-injury. This prolonged time course of disease is consistent with sleep and fatigue symptoms reported in the human condition (Verma et al. (2007) J. Clin. Sleep Med., 3:357-362; Kempf et al. (2010) J. Neurol. Neurosurg. Psychiatry 81:1402-1405). The persistence of symptoms indicates a secondary cascade of effects post-injury that chronically alters brain physiology.

A single mild brain injury also causes profound deficits in brain EEG at a snapshot in time at 2 weeks post-injury. Injured mice show deficits primarily in the dark phase, when mice are typically most active. During the dark phase, injured mice sleep more and are unable to sustain long bouts of wakefulness. They have more fragmented behavioral states and lose the normal diurnal fluctuation in wake bout length. This is consistent with seemingly paradoxical complaints of daytime sleepiness and nighttime insomnia frequently reported in both TBI and narcolepsy patients alike (Verma et al. (2007) J. Clin. Sleep Med., 3:357-362; Castriotta et al. (2010) CNS Drugs 25:175-185; Baumann et al. (2007) Brain 130:1873-1883). In addition, injured mice were unable to sustain wakefulness in situations requiring increased arousal, such as exposure to a novel environment, and showed increased sleep pressure after these short periods of enforced wakefulness. This data indicates that injured mice have less so-called wake reserve, a concept perhaps similar to “cognitive reserve” in aging and neurodegenerative disease (Alexander et al. (1997) Am. J. Psychiatry 154:165-172). It is possible that the widely reported symptoms of fatigue and inattention described after concussion could, in fact, reflect the same mechanisms underlying decreased wake reserve (Dockree et al. (2004) Brain Res. Cogn. Brain Res., 20:403-414).

After injury, there is an overall shift in EEG power density to slower frequencies during the wake state, in particular for theta power. Slowing of theta peak frequency has been implicated in aging and hippocampal-dependent cognitive deficits (Colas et al. (2005) Neurobiol. Aging 26:265-273; Perouansky et al. (2010) Anesthesiology 113:1299-1309). Interestingly, the same left shift in power spectrum is seen in patients with Alzheimer's disease, thought to reflect functional disconnections among cortical areas (Moretti et al. (2011) Front Psychiatry 1:152; Jeong, J. (2004) Clin. Neurophysiol., 115:1490-1505). Similarities in EEG pathology may reflect shared neuropathological mechanisms between TBI and Alzheimer's disease.

Because of similarities in phenotype to human narcolepsy, and human TBI studies implicating dysfunction of the orexin system, alterations in the expression of the neuropeptide orexin was investigated. The involvement of other candidate wake-promoting regions of the brain is less likely given that lesions of such regions (i.e., ventral periaqueductal gray, locus coeruleus, basal forebrain) do not precisely fit the TBI phenotype of sleep fragmentation superimposed on the inability to sustain wakefulness (Lu et al. (2006) J. Neurosci., 26:193-202; Gompf et al. (2010) J. Neurosci., 30:14543-14551; Blanco-Centurion et al. (2007) J. Neurosci., 27:14041-14048). In support of orexin's involvement, several human studies have reported deficits in orexin after TBI. One small study of 4 patients who died 7 to 42 days after severe TBI showed a 27% reduction in the number of orexin neurons compared to non TBI controls (Baumann et al. (2009) Ann. Neurol., 66:555-559). Measured levels of CSF orexin were low in 95% of 44 patients within the first four days of moderate to severe TBI, with the lowest levels of CSF orexin observed in patients who were comatose (Baumann et al. (2005) Neurology 65:147-149). When CSF orexin was measured in these same patients 6 months post-injury, 4 of the 14 patients with excessive daytime sleepiness continued to have low orexin levels, while those without daytime sleepiness showed normalization of CSF orexin levels (Baumann et al. (2007) Brain 130:1873-1883). Controlled-cortical impact (moderate, penetrating brain injury) acutely decreased brain orexin levels in mice, as measured by intracerebral microdialysis within the first three days of injury (Lim et al. (2012) J. Neurotrauma 29:1908-1921). The total number of orexin neurons was unchanged after injury, in contrast to the findings in the small human study, though this likely is explained by differences between mild/moderate versus severe TBI (Lim et al. (2012) J. Neurotrauma 29:1908-1921; Baumann et al. (2009) Ann. Neurol., 66:555-559). The data provided herein indicates that mild/moderate brain injury primarily affects orexin physiology rather than gross cell loss, at least in the immediate weeks following injury.

While the exact mechanisms of injury-induced wake dysfunction are unknown, it is clear that BCAA intervention restores many aspects of wakefulness, including underlying deficits in EEG oscillations and orexin neuron activation. BCAA therapy restores network excitability and hippocampal-dependent cognitive deficits, possibly by restoring pools of releasable vesicular glutamate and GABA (Cole et al. (2010) Proc. Natl. Acad. Sci., 107:366-371). Indeed, glutamate inputs to orexin neurons are well documented to regulate wakefulness, and glutamatergic interneurons have been suggested to play a role in a positive feedback recruitment of orexin on orexin neurons (Acuna-Goycolea et al. (2004) J. Neurosci., 24:3013-3022). In addition, dietary amino acids have been shown to directly affect orexin neuron membrane excitability; this macronutrient sensing mechanism is thought to explain the role of orexin in appetite (Karnani et al. (2012) Neuron 72:616-629).

Branched chain amino acids are essential amino acids and cannot be synthesized de novo, and therefore must be acquired through the diet. Once in the brain, the three BCAA (L-Leucine, L-Valine, and L-isoleucine) are key amino acids involved in de novo glutamate synthesis, as approximately 50% of brain glutamate and 40% of the releasable synaptic glutamate contains BCAA-derived nitrogen (Yudkoff, M. (1997) Glia 21:92-98; Sakai et al. (2004) J. Neurochem., 88:612-622). In a small number of human studies, plasma BCAA levels were decreased with mild and severe TBI, and one follow-up study found that IV BCAA therapy in severe TBI yielded improvement in disability outcomes (Jeter et al. (2013) J. Neurotrauma 30:671-679; Vuille-Dit-Bille et al. (2012) Amino Acids 43:1287-1296; Aquilani et al. (2005) Arch. Phys. Med. Rehabil., 86:1729-1735). Dietary BCAA administration has been studied extensively in healthy people and in disease states over many decades (Fernstrom, J. D. (2005) J. Nutr., 135:1539S-1546S). Patients with a variety of disorders (including liver cirrhosis, bipolar disorder, spinocerebellar degeneration, to name a few), have been treated with BCAA's for variable lengths of time longer than 2 years without adverse effects (Muto et al. (2005) Clin. Gastroenterol. Hepatol., 3:705-713). Overall, BCAA therapy is well tolerated and associated with minimal to no side effects, and therefore is a viable therapy for mild TBI.

### Example 4

Traumatic brain injury affects more than 2.5 million people every year in the United States (CDC). In particular, 50-70% of these injuries are caused by mild TBI (Langlois et al. (2006) J. Head Trauma Rehab., 21(5):375-378). A mild TBI may include a loss of consciousness and/or confusion and disorientation for less than 30 minutes and/or post-traumatic amnesia for less than 24 hours (Carroll et al. (2004) J. Rehab. Med., 43 Suppl: 113-25). Indeed, mild TBI is a difficult disease to treat because patients often recover from the initial injury, but complain of long lasting cognitive sequelae, especially memory deficits (McAllister et al. (2001) Neuroimage 14(5):1004-1012). However, the relation between memory and mild TBI is problematic because it is unknown which aspect of memory is affected by mild TBI.

Decades of research in both humans and rodents has shown that memory is composed of different systems, including episodic memory (Tulving, E. (1985) Elements of Episodic Memory, Oxford Press), semantic memory (Tulving, E. (1972) Episodic and Semantic Memory, Organization of Memory, London: Academic, 381(4):382-404), working memory (Baddeley, A. (1992) Science 255(5044):556), and further sub categories including recollection (Eichenbaum et al. (2004) From conditioning to conscious recollection: Memory systems of the brain (No. 35), Oxford University Press), familiarity (Yonelinas, A. P. (2002) J. Memory Language 46(3):441-517), object memory (Manns et al. (2009) Learning Memory 16(10):616-624), source memory (Glisky et al. (1995) Neuropsychology 9(2):229), and others. Although these categorizations are often debated, sub-dividing memory into smaller sub-categories has proven pivotal to identify the neuro-anatomical basis of the memory system (Zola-Morgan et al. (1993) Ann. Rev. Neurosci., 16(1):547-563). Independent of category, memory is composed of three fundamental phases: encoding, maintenance, and retrieval. Thus, the term memory is a very general concept and is perhaps better thought of as general class of cognition, the specific implementation of which will depend on the category (semantic, episodic, working, etc.) and phase (encoding, maintenance, or retrieval) that is activated. Importantly, different implementations of memory involve different brain regions and neural computations. Analogously, in order to treat the memory deficits caused by TBI, it is important to specifically define the aspect of memory that is affected.

A moderate TBI injury model has shown no deficits during T maze performance when the delay time between sample and choice phase was up to 15 seconds (Lyeth et al. (1990) Brain Res., 526(2):249-258; Whiting et al. (2006) J. Neurotrauma 23(10):1529-1534; Sweet et al. (2014) Hippocampus 24(12):1592-1600). However, increasing the delay time interval, injured animals worsened at successful task performance making more errors compared to their sham counterparts (Whiting et al. (2006) J. Neurotrauma 23(10):1529-1534; Sweet et al. (2014) Hippocampus 24(12):1592-1600; Eakin et al. (2012) J. Neurotrauma 29(6):1180-1187). Work in mild TBI has been less consistent despite the fact that the majority of injuries in people are mild (e.g., concussions). In mild TBI, spatial memory is affected using a Morris water maze (Hylin et al. (2013) J. Neurotrauma 30(9):702-715). However, the delay times on the tasks were very short, and it is unclear if these tasks were testing working memory or episodic memory, which have completely difference neural representations (Zola-Morgan et al. (1993) Ann. Rev. Neurosci., 16(1):547-563; Miller et al. (2006). Neuroscience 139(1):51-58).

Here, a spatial episodic memory task was administered in a mild TBI rodent model and memory performance was tracked across time. In this way, it was established that mild TBI causes a specific problem with episodic memory as well as which particular aspects of episodic memory are affected. Furthermore, it was tested whether BCAA (branched chain amino acids) therapy was able to improve memory function after TBI. BCAA therapy restores the balance between excitation and inhibition in the hippocampus and improve contextual fear conditioning (Cole et al. (2010) Proc. Natl. Acad. Sci., 107(1):366-371; Elkind et al. (2015) Frontiers Neurol., 6:73). Branched chain amino acid therapy also rescues EEG rhythms associated with normal wakefulness after TBI (Lim et al. (2013) Sci. Transl. Med., 5(215):215). Here, the ability of BCAA therapy to mitigate specific spatial memory deficits was tested.

### Material and Methods

### Animals

Experiments were performed on 5-7 week old, 20-25 g, male C57BL/6J mice (Jackson Laboratory Bar Harbor, Me.). All animals were maintained on a 12/12 light-dark cycle, with light on at 8 AM and all experiments were performed during dark cycle. Animals had access ad libitum to food and water. Experiments were designed to minimize the number of animals required.

### Lateral Fluid Percussion Injury

On day 1, all animals were anesthetized with Ketamine (10 mg/100 g body weight; JHP Pharmaceuticals, Parsippany, N.J.) and Xylazine (1 mg/100 g body weight; Phoenix Pharmaceutical, Phoenix, Ariz.) mixture and placed in a mouse stereotactic frame (Stoelting). The scalp was incised and reflected. The following was conducted under ×0.7 to 3.5 magnifications. A craniotomy was performed using a 3 mm trephine over the right parietal area between bregma and lambda, just medial to the sagittal suture and lateral to the lateral cranial ridge. The dura remained intact throughout the craniotomy procedure. A Luer-loc needle hub (3 mm inner diameter) was secured above the skull over the opening with Loctite adhesive and subsequently with cyanoacrylate plus dental acrylic. The Luer-loc needle hub was filled with isotonic sterile saline, and the hub was capped. The mice were, then placed on a heating pad until spontaneous mobile and returned to the home cage. On day 2, the animals were briefly placed under isoflurane anesthesia, the hub was filled with saline and connected via high-pressure tubing to the LFPI device (Department of Biomedical Engineering, Virginia Commonwealth University, Richmond, Va.). The injury was induced by a brief pulse of saline onto the intact dura, which delivers a pressure of 1.5 atm, generating a mild brain injury. Immediately after injury the animals were re-anesthetized with isoflurane for hub removal from the skull and scalp closure. The animals were warmed with an electric heating pad until they could move freely, before they were returned back to their cages and fed with regular diet and tap water. Sham animals received all of the above procedures except the fluid pulse. Each animal was allowed 6 days to recover before behavioral performance.

### Spatial Object Recognition Task

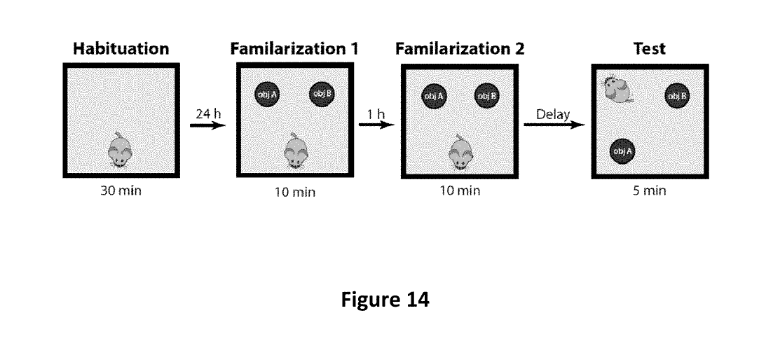

The animals were tested on a memory task that assesses hippocampal-dependent memory for the locations of the objects (Ennaceur et al. (1988) Behavioral Brain Res., 31(1):47-59). As schematically shown in FIG. 14, after a period of 6 days from Injury/Sham procedure, animals were habituated to a square testing apparatus (30 cm×30 cm) with visual cues on the wall for a 30-min period (habituation phase) (Bevins et al. (2006) Nature Protocols 1(3):1306-1311). Item in the recording room were maintained constant across the experiment and served as distal cues, each behavioral experiment consisted of 2 different phases—familiarization (F) and test (T) with rest between them. Specifically, the familiarization phase was split into two sub-phases: Familiarization 1 (F1) and Familiarization 2 (F2). During F1, animals were allowed to explore for a 10-minute period time two identical novel objects located in 2 different quadrant of the apparatus. After F1, the mice rested for one hour before F2 began. During F2, mice were re-exposed to the identical apparatus setting for another 10-min. The next part of the experiment after familiarization was the test phase (T). Before the test phase (T), objects were removed and replaced with another identical pair without any marks making them undistinguishable. One object was left in the previous location and the other object was placed in a previously empty quadrant. The test phase lasted 5 minutes, during which time the animals explored the new apparatus setting. The test phase tested the ability to identify the new location in the already well-known environment. The analysis was restricted to the first 3 minutes of the test phase to avoid habituation phase.

One goal of this experiment was to explore how memory performance changes across time. To do that, the time between familiarization (F) and test (T) was varied. Specifically, three different delay times between familiarization (F) and test (T) were used: 3 minute, 1 hour and 24 hours. Animals were randomly assigned to each of these groups. All analyses were made with experimenter blinded to both experimental conditions: (1) injury vs. sham and (2) the delay time between familiarization and test.

Exploratory behavior was quantitated as the time the animal spent with its nose directed toward any object within a distance of <2 cm. Of note, other behaviors such as sitting on top of the object, looking around while sitting or resting against the object were not considered exploratory behavior. Any animal with less than of 15 seconds exploratory behavior during familiarization, or with less than 10 seconds of exploratory behavior during test, were excluded from all analyses. Novelty preference was defined as the absolute difference in time spent exploring the novel and the familiar object divided by the total time spent exploring both objects. A preference score of 1 indicated that animals explored only the object in the new location, a score of 0 denoted no preference or equal time exploring the object in the new and old location and a score of −1 indicated that the animal explored only the object in the familiar location.

### Treatment

In order to test the effect of the branched amino acids (BCAA) therapy on the memory task performance, an additional group of injured animals (N=11) was treated with BCAA dietary therapy. Specifically drinking water was supplemented with 100 mM of leucine, isoleucine and valine amino acids and consumed ab libitum. Animals started the treatment 48 hours after injury and continued until the test phase of the task. As described before, animals were allowed to recover for 6 days and then perform the behavioral paradigm with 1 hour delay time between familiarization and test.

### Statistical Approach

Data collection and analysis were performed blinded to experimental group. Statistical analyses were performed using custom software written in Matlab (Mathworks). Exploratory behavior was quantified as the time the mouse spent with its nose directed toward any object within a distance of <2 cm. Of note, other behaviors such as sitting on top of the object, looking around while sitting or resting against the object were not considered exploratory behavior (Bevins et al. (2006) Nat. Prot., 1(3): 1306-1311). Any mouse with less than 15 seconds of exploratory behavior during familiarization, or with less than 10 seconds of exploratory behavior during test, were excluded from all analyses. Novelty preference was quantified with the Discrimination Index (DI) which was defined as the absolute difference in time spent exploring the novel and the familiar object divided by the total time spent exploring both objects. A preference score of 1 indicated that mice explored only the object in the new location, a score of 0 denoted no preference or equal time exploring the object in the new and old location and a score of −1 indicated that mice explored only the object in the familiar location.

The discrimination index (DI) was found for each mouse during the three testing intervals: 3-minutes, 1 hour and 24 hours. In addition, the percentage of time during F1, F2, and T that each mouse spent in exploratory behavior (EB) was calculated. These DI and EB measures were compared across TBI and Sham using a two-tailed unpaired Student's t-test. The EB values were also calculated across time during F1 and F2 (FIG. 3). Specifically, F1/F2 was segmented into 90 second sliding windows with 15 seconds increment (80% overlap). The percentage of time in EB in each time window was found and compared across time for Sham and TBI. An identical analysis with 200 second, non-overlapping windows during F1/F2 was also run. This allowed a two-way ANOVA to be performed on the F1 and F2 data, treating the time windows and experimental condition (TBI vs Sham) as independent factors.

### Results

It was investigated if a particular memory component (encoding, maintenance, retrieval) was disproportionately impaired after mild TBI. To do that, 58 mice were run in a spatial memory task, half of which were subjected to a mild TBI using a lateral fluid percussion injury model (LFPI). Briefly, the spatial memory task (see FIG. 14) consists of three phases: Habituation (H), Familiarization (F), and Test (T). During Familiarization, mice were freely exposed to a platform with 2 identical objects across two separate 10-minute blocks (F1 and F2). Subsequently, during the test phase (T), one of the objects was randomly moved to a previously empty quadrant and we tested the animal's ability to identify the new location in the already well-known environment. To measure memory performance as a function of time, a variable delay time between F2 and T was interlaid.

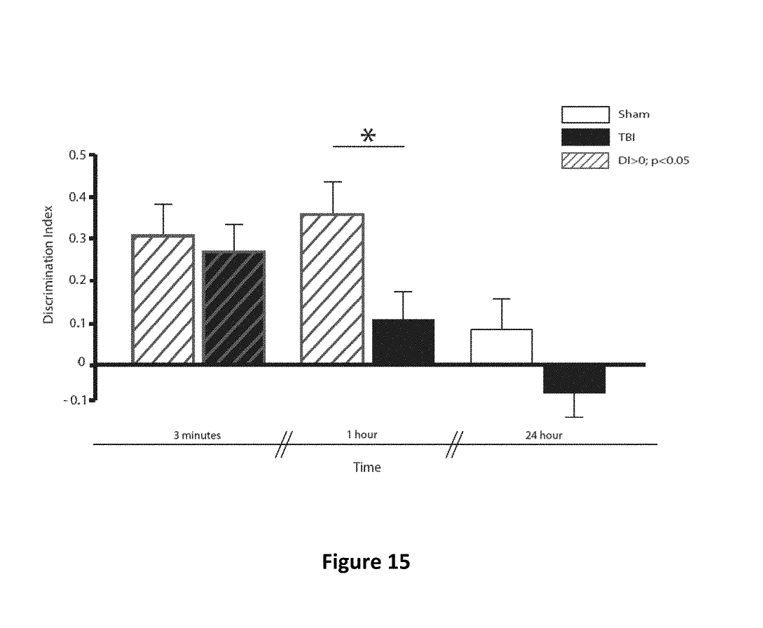

Using this task, memory performance was quantified across time using the discrimination index (DI), which measured whether the mice preferentially explored the novel location during test (FIG. 15). Specifically, TBI vs Sham DI was compared for all three sets of time delays. It was found that there was a significant increase in DI above chance for both TBI and Sham at 3 minute delay (T₍₉₎=3.10, p<0.05 for Sham, T₍₉₎=4.89, p<0.05 for TBI). However, only Sham also had a significant increase in DI above chance after one hour (T₍₉₎=4.53, p<0.05). Furthermore, the difference in DI between TBI and Sham at the one-hour delay time was significant (T₍₁₈₎=2.36, p<0.05). Neither TBI nor Sham had a significant increase in DI at the 24 hour time delay (p>0.2). These data indicate that both TBI and Sham mice successfully retrieved object location after a 3 minute delay. However, TBI animals showed faster memory decay than Sham animals. Specifically, injured animals “forgot” object location after 1 hour, whereas sham mice “forgot” objection location after 24 hours.

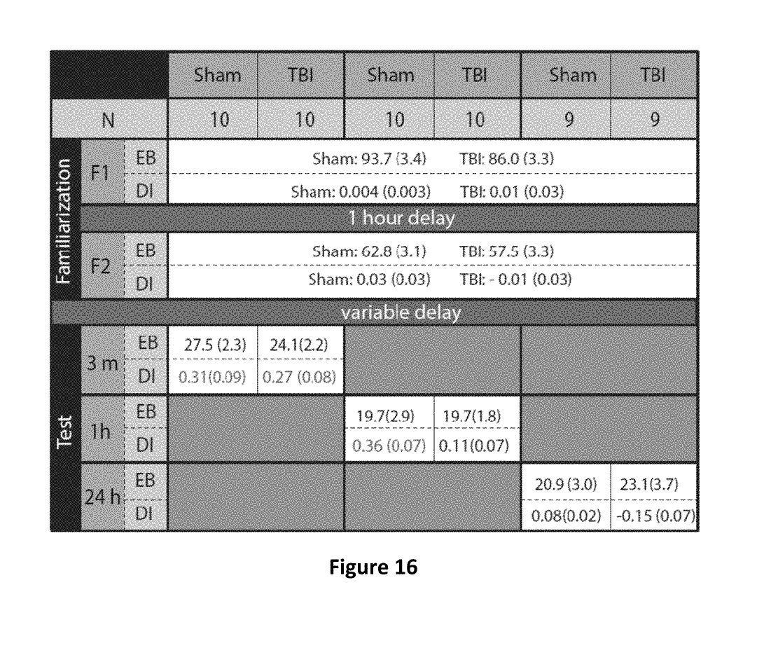

The previous result indicates that injured mice have a deficit in spatial (episodic) memory. However, it is unclear if this deficit involves encoding, maintenance, or retrieval. To investigate if episodic encoding is affected in TBI, the exploratory behavior of mice as analyzed when they were first exposed to the objects, i.e. during the first round of familiarization (F1). Specifically, the time spent in exploratory behavior (EB) during F1 was measured. When analyzing the F1 period, animals were collapsed across F1-T delay time, leaving 58 animals: 29 TBI animals and 29 Sham animals. The time spent in EB during F1 for both TBI and Sham was compared. It was found that there was no difference in overall EB between TBI and Sham across the entire 10-minute F1 interval (p>0.2) and DI was not different than chance (p>0.2) (FIG. 16).

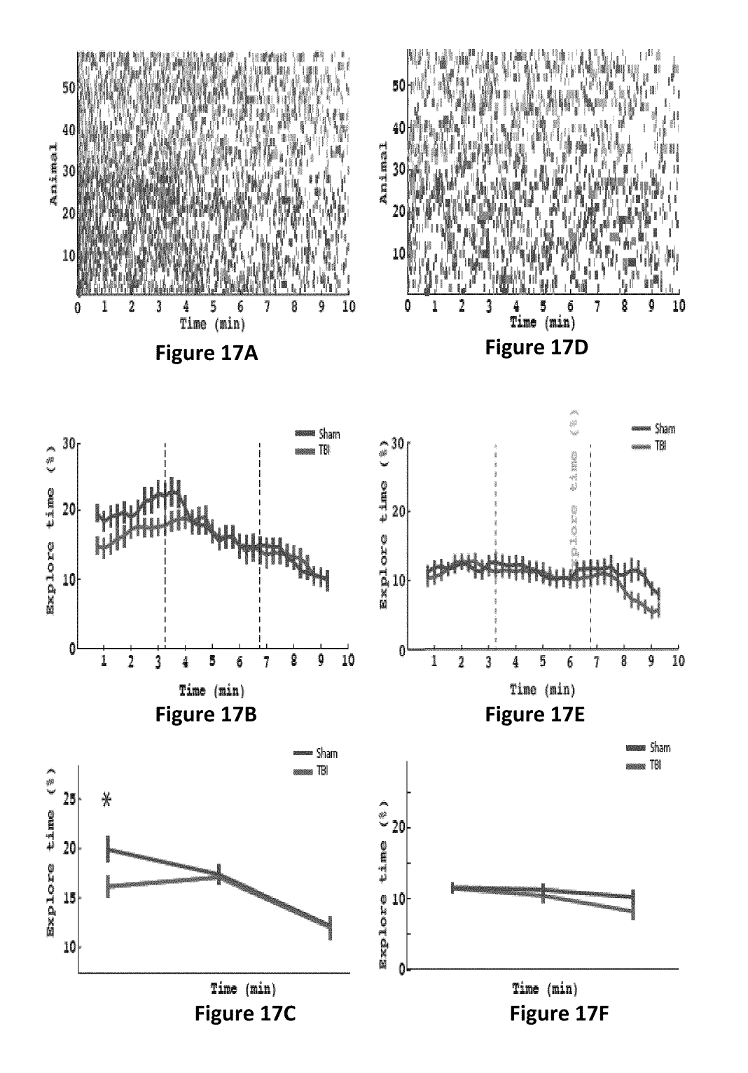

It was hypothesized that the episodic encoding interval represented a relatively discrete amount of time when the animals first encountered the objects. Thus, EB was analyzed as a function of time during F1. FIG. 17A represents the entire behavioral data for all animals during F1: each row represents an animal, and each color represents a discrete episode of EB during F1. FIG. 17B collapses this information separately across TBI and Sham animals by averaging, across animals, the percentage of time spent in EB for a series of 90-second time epochs incremented every 15 seconds throughout F1. From FIG. 17, Sham animals spend a disproportionate time in EB very early in F1, during the hypothetical episodic encoding window. To test this, two actor ANOVA was constructed assessing the EB across both injury condition and time during F1. For this analysis, three time windows were defined during F1: early (0 to 3.3 minutes), middle (3.4 to 6.6 minutes) and late (6.7 to 10 minutes). A significant main effect was found across time (F_{2,198}=26.32, p<0.001) and an interaction effect between time and injury condition time (F_{4,198}=2.59, p<0.05). Post-hoc t-test revealed that the interaction effect was driven by a difference in EB during the first time window in the F1 time epochs (p<0.05). Together, these data indicate that TBI mice explore object location less than Sham mice specifically during the episodic encoding time window and also later have a decrease ability to recall object location during the test phase.

The F2 time period was also examined to determine if the increase in EB seen during F1 is also seen during F2. FIGS. 17D-17F show the same analyses repeated for F2. Of note the increase in EB seen during the first part of F1 is not seen during F2. More specifically, the same ANOVA applied to the F2 time interval did not show a main effect across time, injury condition, or an interaction effect. These data indicate that the increase in EB seen for Sham mice but not for TBI mice during the F1 interval does not represent a generic exploration initialization signal, rather it is specific to the very first time the animal interacts with the objects consistent with an episodic encoding signal.

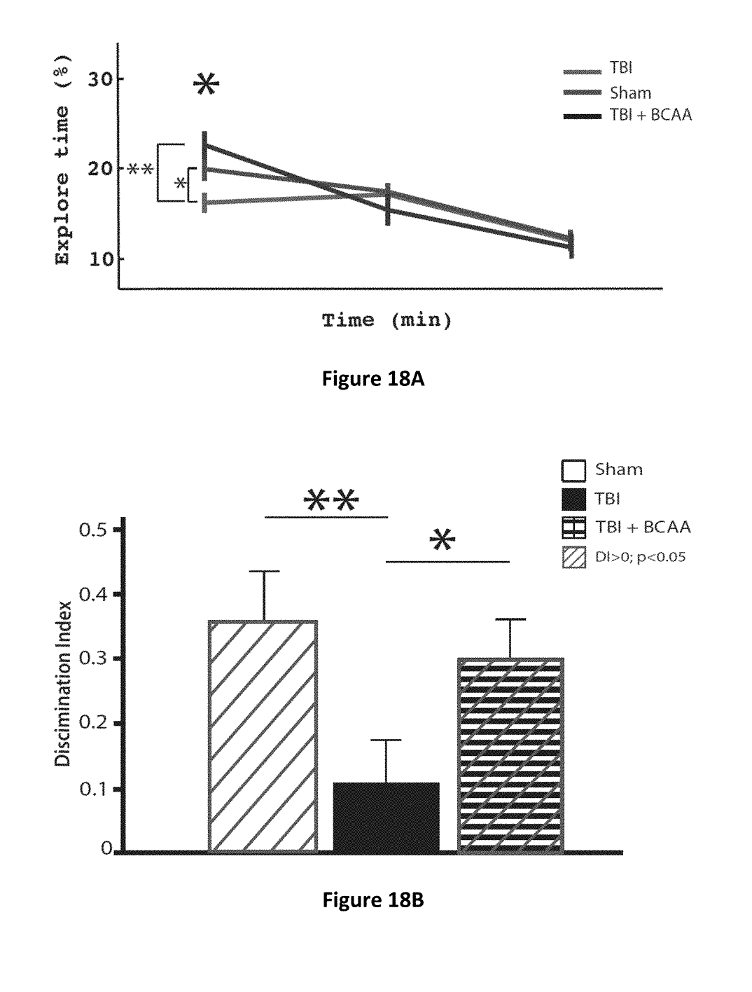

It has been shown that TBI leads to a decrease in EB during the initial part of F1 and a decrease ability to retrieve spatial information after a long delay during Test. If the decrease in EB during F1 represents a deficit in episodic encoding, then interventions that reverse the deficit in memory performance during test should also reverse the decrease in EB during F1. To test this, BCAA therapy was administered to TBI animals. BCAA therapy has been shown to improve contextual fear conditioning in TBI animals after injury indicating that it may improve memory in TBI animals. Here, separate groups of TBI animals (n=11) and Sham (n=8) were isolated and these mice were started on an enriched dietary therapy with BCAA (100 mMol) 48 hours post-injury, which continued up to the test day. Animals were tested after 1 hour delay time between the second familiarization and test. It was found that BCAA therapy improved the animal performance during the test phase of task, i.e. BCAA mice had a significant increase in DI during test (T₍₁₀₎=4.42, p<0.05) that was significantly greater than the DI for the TBI animals (T₍₁₉₎=1.96, p=0.06) (FIG. 18). Furthermore, BCAA mice also had a specific increase in EB during the first three minutes ofF1 compared to TBI mice (p<0.05). In contrast, Sham-BCAA mice showed a decreased ability to discriminate spatial location after 1 hour delay. Taken together, these data show that reversing the memory deficit caused by TBI also causes an increase in exploratory behavior during episodic encoding.

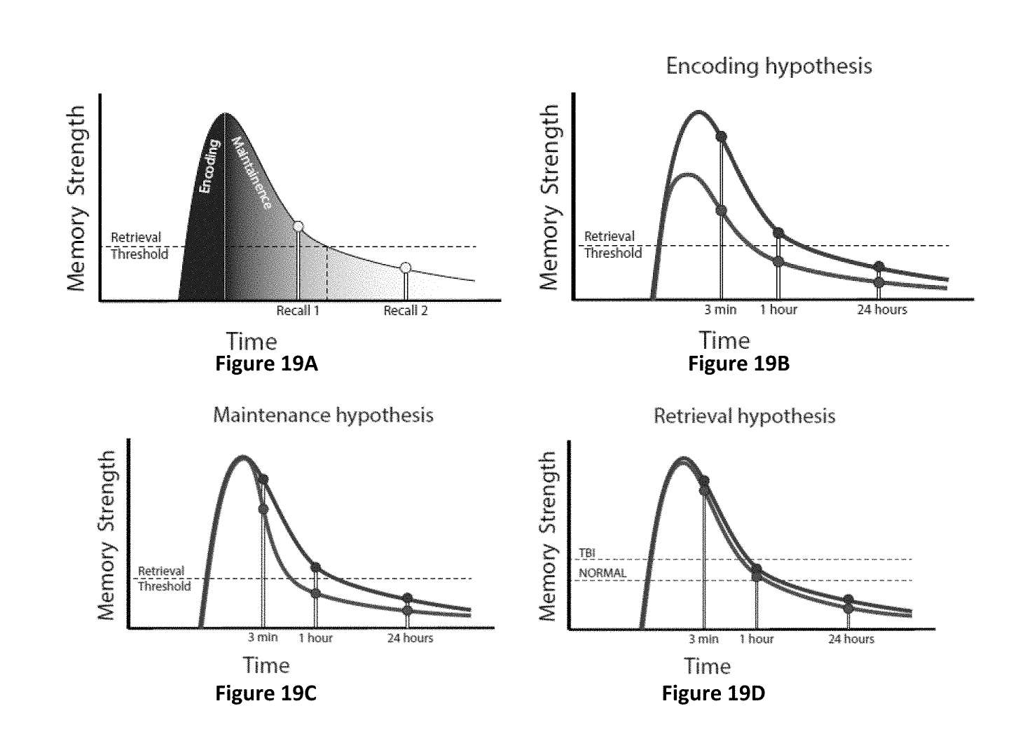

The affect of mild TBI on spatial episodic memory was investigated. To do this, 58 mice were subjected to a spatial episodic memory task, half of which endured a mild TBI. It was found that TBI had two distinct effects on task performance: (1) TBI mice explored less during the initial episodic encoding interval and (2) TBI mice forgot spatial information much more quickly than controls. Given that episodic memory is composed of encoding, maintenance, and retrieval, there are three independent mechanisms that could explain these data, which are outlined in FIG. 19 and discussed below.

In FIG. 19A, a strength theory view of episodic memory is briefly outlined. Strength theory is a simple and well-accepted model of human episodic memory (in particular recognition memory) (Kahana, M. J. (2012) Foundations of human memory. Oxford University Press), and is based on the premise that each item has a numerical value representing how well the item triggers a memory response (CITE). In FIG. 19A, one item's strength is shown across time. With regard to the current task, an item represents the combined information about one object (spatial location, object information, and current context). During encoding, the memory strength for that object increases rapidly while the animal explores the object. During the maintenance period, the item is not encountered and the strength gradually decreases over time, i.e. the item is forgotten. Finally, during retrieval, if the item's strength is above a threshold then the item can be recalled with perfect fidelity (Recall 1), or if the memory strength is below the retrieval threshold the item is forgotten (Recall 2). Of course, this model is a simplification of actual memory dynamics and does not take into account different types of item information (spatial, object, contextual), variation in retrieval success, etc. However, it is a useful heuristic to understand memory dynamics.

In FIGS. 19B-19D, three possible mechanisms of episodic memory failure after TBI are outlined. First, TBI could lead to a deficit in encoding (Encoding hypothesis, FIG. 19B). In terms of strength theory, this would be represented by a decrease in the strength of the item during the initial encoding interval. Alternatively, TBI could cause a problem of memory maintenance (Maintenance hypothesis, FIG. 19C), causing injured animals to have a faster decay period (i.e. faster forgetting rate) then Sham animals. Finally, TBI animals could have a problem in memory retrieval (Retrieval hypothesis, FIG. 19D) and require more memory strength for each individual item to trigger a memory event. Like other pathologies of memory (Alzheimer's Disease, cognitive decline of aging, etc.), TBI may be a global problem leading to a deficit in encoding, maintenance, and retrieval. However, these processes are theoretically independent and have different electrophysiological signatures (Montgomery et al. (2007) PNAS 104(36):14495-14500; Carr et al. (2011) Nature Neuroscience 14(2):147-153).

Here, the data indicates that TBI disproportionately affects episodic encoding for the following reasons. First, there is a decrease in exploratory behavior (EB) during the first three minutes in F1 (see FIG. 17A-17C), which is the time period that most likely represents pure episodic encoding because there is no previous object memory to retrieve. Second, there is no difference in EB between TBI and Sham during F2 after a one-hour delay (see FIGS. 17D-17E). This indicates there is some degree of maintenance/retrieval of the objects after one hour. Third, therapies that reverse the memory deficit after a one-hour delay also increase exploratory behavior during F1 (see FIG. 18). These data indicate that EB during F1 directly correlates with changes in memory performance, consistent with the hypothesis that TBI affects episodic memory by causing a deficit in encoding. If this is the case, then interventions that reverse the deficit in memory performance should also reverse the decrease in EB during F1. Therefore, the memory deficits after TBI were reversed with BCAA therapy, and, consistent with the episodic encoding postulate, we found that the effect of EB was also reversed. Taken together, these data show that reversing the TBI-related deficit in episodic encoding (as reflected by and perhaps caused by the deficiency in exploratory behavior) is associated with a rescue of the memory deficit.

Significantly, it is also shown herein that branched chain amino-acid reverses spatial memory deficits after TBI. BCAA therapy also reverses contextual fear condition memory task after 7 days post injury. Indeed, BCAA therapy also reverses injured-induced shift in net synaptic efficacy (Cole et al. (2010) PNAS 107(1):366-371). Branched chain amino acids (BCAAs) serve a dual role in the CNS. In addition to the fundamental use of amino acids for protein synthesis, these specific amino acids (valine, leucine and isoleucine) are pivotal in glutamate synthesis since they contribute 50% of the nitrogen in glutamate (Kanamori et al. (1998) J. Neurochem., 70(3):1304-1315; Sakai et al. (2004) J. Neurochem., 88(3):612-622). Furthermore, de novo glutamate synthesis contributes a significant amount (approximately 40%) of releasable synaptic glutamate (Lapidot et al. (1994) J. Biol. Chem., 269(44):27198-27208; Lieth et al. (2001) J. Neurochem., 76(6):1712-1723).

TBI induced by lateral fluid percussion leads to a significant reduction in ipsilateral BCAA concentration which can be restored by dietary therapy. Furthermore, BCAA therapy for 5 days administered 48 hours post injury rescue contextual fear conditioning memory after 7 days post injury highlighting the potential neuro-restorative role of this therapy. Indeed, BCAA therapy as well as in vitro BCAA application has been shown to also reverse injury-induced shifts in net synaptic efficacy as well as an inability to maintain wakefulness.

TBI affects EB during familiarization. Here, this decrease in EB represents a decrease in episodic encoding in TBI mice. The link between EB and episodic encoding is supported in the extant literature, especially in electrophysiological studies. In rodents, exploratory and oriented-volunteer behavior are known to be related with theta oscillations in the hippocampus (Vanderwolf, C. H. (1969) Electroencephalography Clin. Neurophysiol., 26(4):407-418.). Theta oscillations facilitate the formation of maps and episodic memory (Buzsaki, G. (2005) Hippocampus, 15(7):827-840; Nyhus et al. (2010) Neurosci. Biobehav. Rev., 34(7):1023-1035). Indeed, theta oscillations are one of the most well studies electrophysiological markers of encoding (Buzsaki et al. (2013) Nature Neurosci., 16(2): 130-138). Thus, the data support a link between episodic encoding and EB, which is consistent with the hypothesis that TBI disproportionally affects memory encoding.

### Example 5

Cognitive impairment caused by traumatic brain injury (TBI) can lead to devastating consequences for both patients and their families. The underlying neurological basis for TBI-induced cognitive dysfunction remains unknown. However, many lines of research have implicated the hippocampus in the pathophysiology of traumatic brain injury. In order to investigate the effects of traumatic brain injury on specific cognitive dysfunction involving the hippocampus, the lateral fluid percussion injury animal model was used and in vivo electrophysiological activity in the hippocampi of mice was recorded. Specifically, local field potentials in areas CA1 and CA3 of the hippocampus were simultaneously measured while animals were performing a spatial object recognition task in TBI and Sham animals. In vivo electrophysiological recordings demonstrated an increased theta activity in both CA1 and CA3 during exploration versus resting in TBI and in Sham animals. Furthermore, it was found that the overall broadband power in area CA1 and CA3 was decreased in TBI animals compared to their counterpart Sham control group during exploration of a new object compared to resting activity. Also, differential activity in CA1 versus CA3 was found in a complex interaction depending on behavior performance between TBI animal versus Sham control group. Those findings indicated for the first time that both area CA1 and CA3 of the hippocampus are impaired after TBI and further explores the neural basis underlying cognitive dysfunction.

### Materials and Methods

### Fluid Percussion Injury (FPI)

After craniotomy, a Luer-loc needle hub (3 mm inner diameter) was secured above the skull opening. On the next day, the hub was filled with saline and connected via high-pressure tubing to the FPI device. The injury was induced by a brief pulse of saline onto the intact dura. Sham animals received all of the above except the fluid pulse. Subsequent experiments were performed 6-8 days after.

### Electrophysiology

All experiments were performed on 8 to 12 week old C57/BL6 mice (Jackson Laboratory, Bar Harbor, Me.). Mice were anesthetized with ketamine/xylazine, and 4 independently movable tetrodes were guided into the hippocampal area CA1 and CA3 using the stereotaxic coordinates. Reference and ground electrode was placed in the contralateral hippocampus and in the cerebellum respectively with 2 screws. The entire ensemble was secured with dental cement (Plastick One, Roanoke, Va.). Following surgery, all animals were allowed to recover under a heat lamp or on a servo-controlled heating pad, until spontaneously mobile. The animals were then returned to their home cage for more than 4 days before being hooked up to the recording apparatus. Animals (sham and LFPI) were recorded for the duration of the object-place task. All recordings were performed using a Neuralynx Cheetah A/D system, with a passband filter setting of 0.1-9 kHz. Sampling rates 32 kHz.

After the recordings, all animals were sacrificed and processed for histological assessment of the electrodes. The electrode position was marked by passing 20μ for 20 seconds through each wire. Electrode locations were verified by microscopic analysis of tissue counter-stained with Prussian Blue.

### EEG Analysis

Each EEG was reviewed to characterize the amplitude and frequencies in hippocampal electrodes during the task performance. Power spectra were calculated for each EEG segment analyzed, using custom written software.

### Behavior

Animals performed object-place recognition task to investigate hippocampal memory dysfunction after LFPI. Mice were handled for 5 minutes per day for 3 days before habituation phase of the task. They were habituated for 30 minutes to an open field (35×35 cm) with a local cue in 2 opposite walls. The following day, animals performed the familiarization and the test with a delay (3 minutes, 1 hour or 24 hours). In all animals the test was performed after 7-8 days post injury.

### Results

In vivo hippocampal electrophysiological recordings showed an overall decrease in broadband power in area CA3 across all time periods during the first familiarization and an impaired broadband corrected theta power in area CA1 during exploration of the 2 new objects (encoding phase of memory).

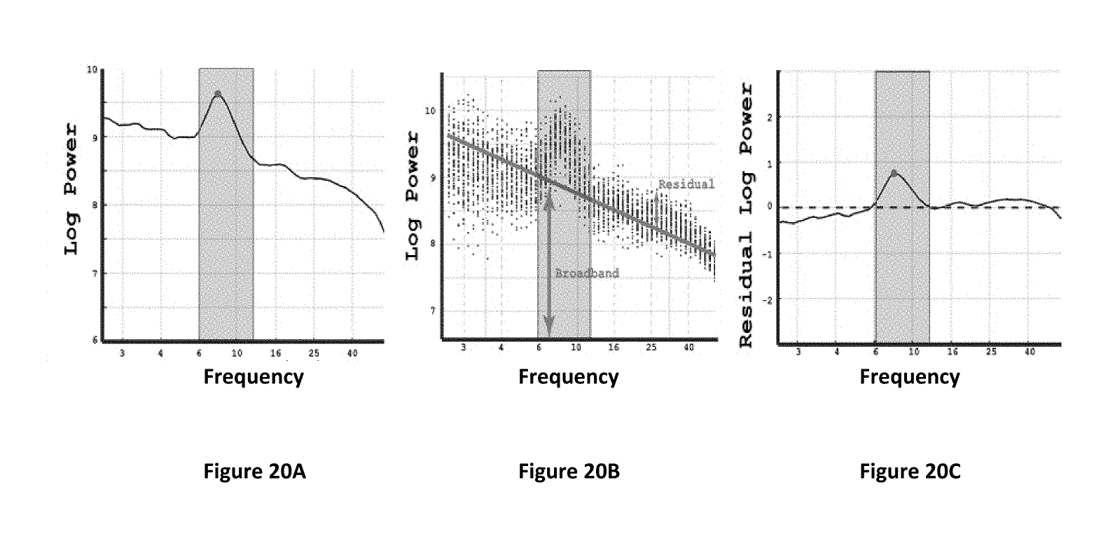

FIG. 20 provides a schematic representation of broadband correction. FIG. 20A provides an averaged power spectrum of one animal in the sham condition. FIG. 20B provides the distribution of power values across all time epochs. The line represents a regression through the distribution of power values. The broadband power is represented by the height of the line. The residuals (regression line subtracted from the power values) represent the broadband correction. FIG. 20C shows the broadband corrected power spectra for the same sham animal.

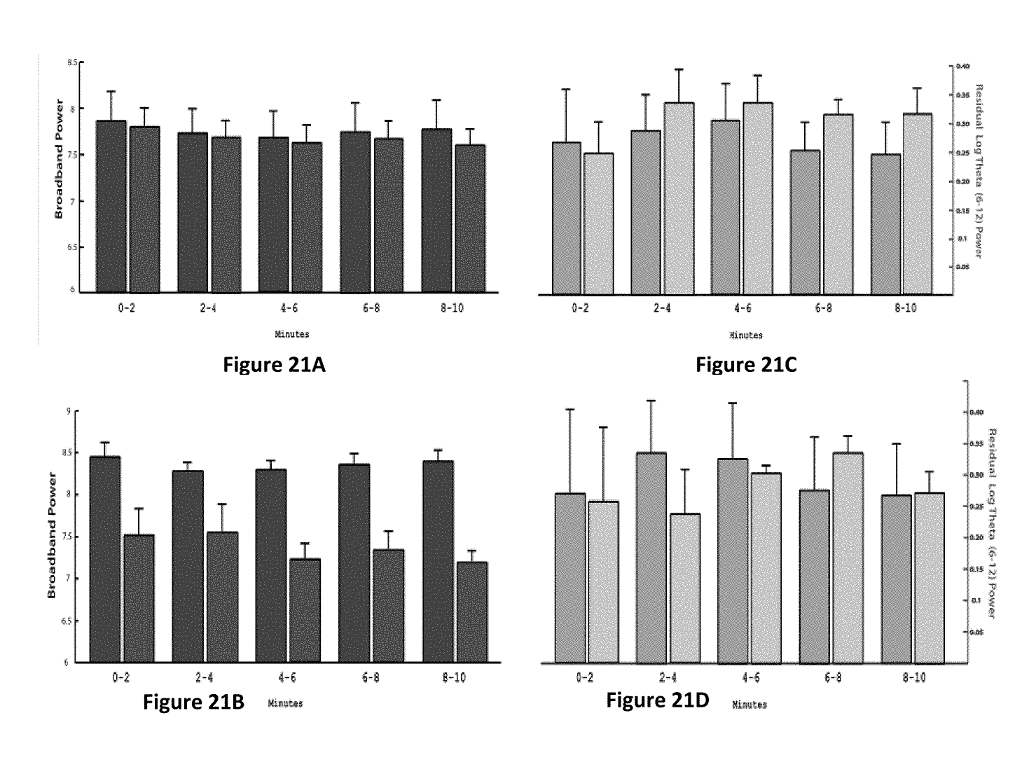

FIGS. 21A and 21B provide the averaged total broadband power in sham and TBI animals segmented into 120 second time windows during the first familiarization in area CA3 and CA1. FIGS. 21C and 21D show the averaged broadband corrected residual theta power in sham and TBI animals segmented into 120 second time windows with 5% overlap during the first familiarization in area CA3 and area CA1.

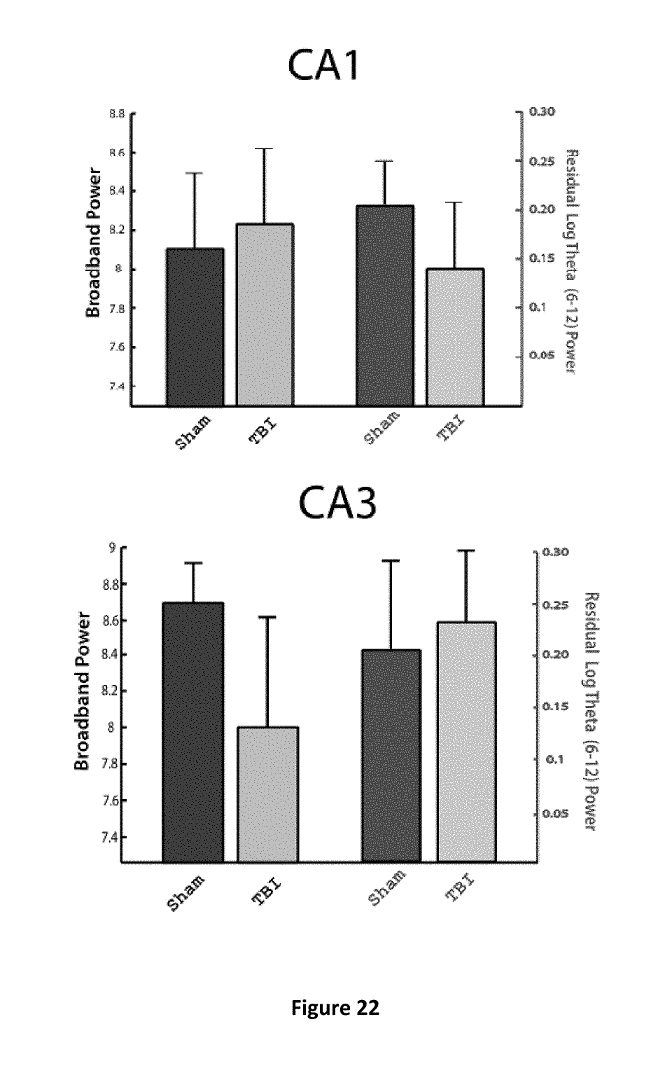

FIG. 22 shows the averaged total broadband power and averaged broadband corrected residual theta power in sham and TBI in area CA3 and CA1 during the exploration objects time windows.

### Example 6

Mild traumatic brain injury (mTBI) leads to an array of long-lasting cognitive symptoms, notably including episodic memory dysfunction (Tsirka et al. (2010) Int. J. Neurosci., 120:184-191). In the healthy brain, episodic memory relies on cortical input of spatial information to the hippocampus for memory encoding and consolidation. Experimental mTBI models have specifically demonstrated that hippocampal disruption is associated with spatial memory impairments (Zohar et al. (2011) Acta Neurobiol. Exp., 71:36-45; Tweedie et al. (2013) Exp. Neurol., 239:170-182; Witgen et al. (2005) Neuroscience 133:1-15; Milman et al. (2005) J. Neurotrauma 22:1003-10). The dentate gyrus is poised to receive incoming signals from layer II/III entorhinal cortex via perforant path axons and transmit signals to the CA3 region of the hippocampus via the mossy fiber axonal pathway (Witter, M. P. (2007) Prog. Brain Res., 163:43-61; Witter, M. P. (2007) Learn. Mem., 14:705-713). Synaptic transmission through this di-synaptic pathway plays a critical role in the processing of spatial information received by the hippocampus, and in pattern separation and completion (Marr, D. (1971) Source Philos. Trans. R. Soc. London. Ser. B, Biol. Sci. 262:23-81; McNaughton et al. (1987) Trends Neurosci., 10:408-415; O'Reilly et al. (1994) Hippocampus 4:661-682; Treves et al. (1994) Hippocampus 4:374-391; Leutgeb et al. (2007) Science 315:961-966; Bakker et al. (2008) Science 319:1640-2; Gilbert et al. (2001) Hippocampus 11:626-636; Hunsaker et al. (2008) Hippocampus 18:1064-1073; McHugh et al. (2007) Science 317:94-9; Nakashiba et al. (2012) Cell 149:188-201).

The dentate gyrus (DG) is thought to act as a physiological gate, or filter, of afferent input into the hippocampus, limiting afferent cortical excitation to area CA3 (Hsu, D. (2007) Prog. Brain Res. 163:601-613). This filtering property is distinctly exhibited by the sparse, action potential firing of dentate granule cells in response to perforant path stimulation (Andersen et al. (1966) Acta Physiol. Scand., 66:448-460; Alger et al. (1976) Brain Res., 110:463-480; Teyler et al. (1976) Brain Res., 115:413-425). Sparse granule cell activation is likely due to both the low intrinsic membrane excitability of granule cells, as well as robust GABAergic inhibition from interneurons within the DG network (Coulter et al. (2007) Prog. Brain Res., 163; Staley et al. (1992) J. Neurophysiol. 67:1346-1358; Fricke et al. (1984) J. Neurophysiol. 51:195-209; Scharfman, H. E. (1992) Epilepsy Res. Suppl. 7:93-109; Williamson et al. (1993) Brain Res., 622:194-202). As such, the putative dentate filter may be functionally important for discerning relevant spatial information for further hippocampal memory processing and diminishing irrelevant inputs.

Lateral fluid percussion injury (LFPI) is a well characterized and commonly used rodent model of brain injury that reproduces key features of human TBI, including neuronal cell loss, gliosis, ionic perturbation, and memory deficits (Dixon et al. (1987) J. Neurosurg., 67:110-119; McIntosh et al. (1989) Neuroscience 28:233-244; Smith et al. (1991) J. Neurotrauma 8:259-269). After LFPI, the DG experiences a net increase in network excitability associated with an imbalance of excitatory and inhibitory synaptic transmission onto dentate granule cells (Santhakumar et al. (2000) J. Physiol., 524 Pt 1:117-34; Toth et al. (1997) J. Neurosci., 17:8106-17; Lowenstein et al. (1992) J. Neurosci., 12:4846-4853; Santhakumar et al. (2001) Ann. Neurol., 50:708-717). Post-traumatic alterations in individual DG cell types have been well characterized. Spatial novelty detection impairments occur in a hippocampal-dependent behavioral task at one week after LFPI. To evaluate DG filtering efficacy, electrophysiological techniques in acute brain slices was used to assess network excitability in DG and area CA3 in response to perforant path stimulation. The results demonstrate that afferent excitation not only generated a hyperexcitable shift in the DG network response after LFPI, but was also accompanied by aberrant propagation of excitation to CA3 and increased CA3 network activation due to mossy fiber synaptic transmission. Thus, synaptic transmission through the dentate gyrus to area CA3 is altered after LFPI, indicating diminished filtering capacity of the dentate gyrus in the injured brain.

### Materials & Methods

### Fluid Percussion Injury (FPI)

Anesthetized, male C57/Bl6 mice ages 6-8 weeks underwent surgery to receive a craniectomy of the right parietal bone. After craniectomy, a Luer-loc needle hub (3 mm inner diameter) was secured above the skull opening. The injury was induced by a brief pulse of saline (1.5 atm) onto the intact dura. Sham controls underwent all surgical procedures except the fluid pulse. Subsequent behavioral testing was performed 7 days after injury. Experimenters were double-blinded to all injury conditions during behavior and analysis.

### Behavior

Mice performed a 3 object-spatial object recognition task. Mice were handled for 5 minutes per day for 4 days before training. During the familiarization day, mice received one 6 minute habituation and three identical 6 minute training trials. Mice were allowed to freely explore the environment and the objects for the duration of each trial. After 24 hours from the first training trial, mice were placed back in box for the testing phase that also spanned 6 minutes. The three objects were again present, but one of the three objects was now displaced to a novel spatial location. Time spent exploring the displaced and non-displaced objects was measured.

### BCAA Treatment

Drinking water in the treatment groups was supplemented with 100 or 50 mM concentrations of each of leucine, isoleucine, and valine and consumed ad libitum (time course, duration of action, and supplement concentration experiments). Control treatment consisted of drinking untreated tap water. Mice drank on average 3-5 mL of solution per day and it has been shown that BCAAs do not affect body weight.

### Analysis

The response to spatial change (discrimination index) was measured by comparing the percent of total object interaction time the mice spent exploring the objects belonging to each category (displaced and non-displaced) in the test session minus the percent time exploring the same object category in the third training session. A positive value indicates recognition of the spatial change. Criteria for what was considered as object interaction was limited to the times when mice were facing and sniffing the objects within a 2 cm radius and/or touching them; sitting over the objects did not count as an exploration. Researcher was blinded to the animal groups during behavior and subsequent analysis.

### Results

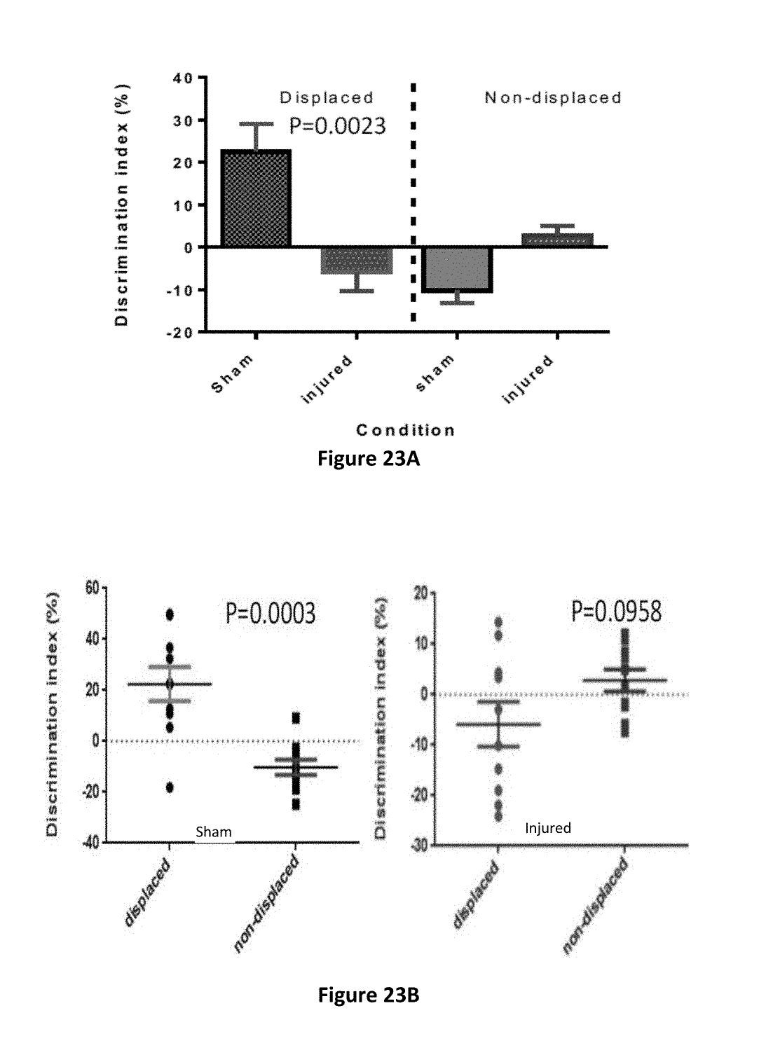

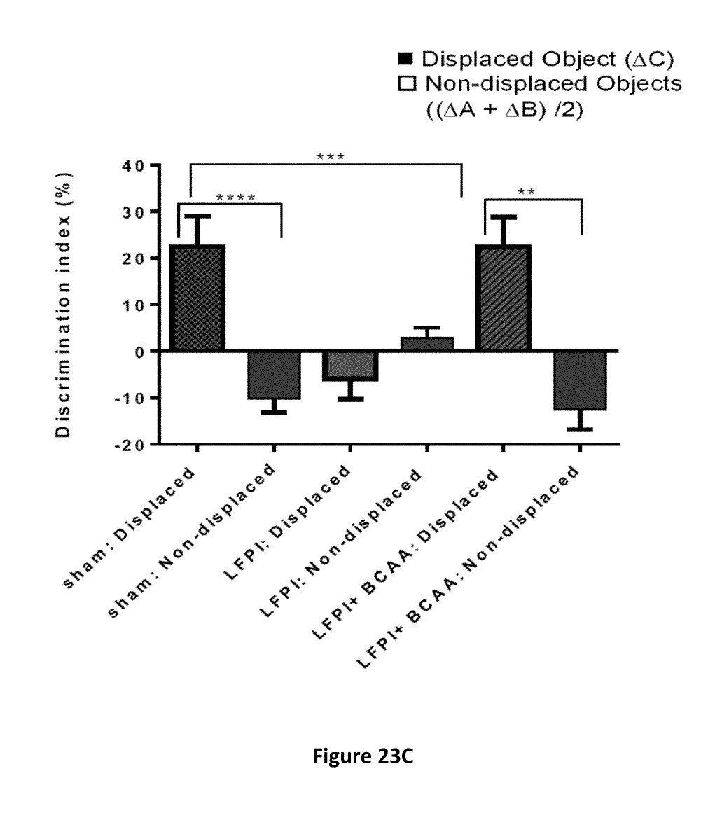

FIG. 23A provides the discrimination index of both the sham and injured groups using the first 2 minutes of the third training and testing trials. There is a significant difference between the ability of sham and injured animals to discriminate the displaced object. Sham mice were able to easily discriminate that one of the objects was displaced, but FPI mice were unable to discriminate. FIG. 23B shows the discrimination index for sham and injured groups separately to show variance. FIG. 23C shows that BCAA administration improves discrimination ability after mTBI. The graph includes sham, LFPI and BCAA group. Results show that BCAA restores spatial performance. All values were statistically significant (P value<0.001***).

Thus, injured mice exhibited impaired discrimination of the displaced object in this dentate-involved behavioral task compared to the sham group whose discrimination was intact. Administration of BCAA for one week after injury restores pattern separation ability in LFPI mice. This indicates that the observed physiological abnormalities (increased excitability) in the dentate gyrus as a result of mild TBI translate into behavioral deficits and that BCAA ameliorates these behavioral impairments. This is consistent with the finding that BCAA can restore shifts of synaptic excitability after mTBI.

A number of publications and patent documents are cited throughout the foregoing specification in order to describe the state of the art to which this invention pertains. The entire disclosure of each of these citations is incorporated by reference herein.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method for reducing a cognitive impairment in a subject, said method comprising administering to said subject at least one branched chain amino acid.

2. The method of claim 1, wherein said cognitive impairment is a memory deficiency.

3. The method of claim 2, wherein said memory deficiency is a deficiency in encoding.

4. The method of claim 1, wherein said method comprises administering valine, leucine, and isoleucine to said subject.

5. The method of claim 1, wherein said branched chain amino acid is in a composition with at least one pharmaceutically acceptable carrier.

6. The method of claim 1, wherein said cognitive impairment is associated with a brain injury.

7. The method of claim 1, wherein said subject has a disease, disorder, or condition selected from the group consisting of Alzheimer's disease, Parkinson's disease, Lewy-bodies dementia, dementia, chronic traumatic encephalopathy (CTE), dyslexia, attention-deficit/hyperactivity disorder (ADHD), stroke, traumatic brain injury, asphyxia, cardiac arrest, coma, meningitis, and epilepsy and status epilepticus.

8. The method of claim 1, further comprising administering acetate to said subject.

9. The method of claim 1, wherein said branched chain amino acids are administered orally or nasally.

10. The method of claim 2, wherein said memory deficiency is a deficiency in spatial memory.

11. The method of claim 1, wherein said method comprises administering to said subject a composition comprising valine, leucine, isoleucine, a flavor masking agent, a sweetener, and a flavoring agent.

12. The method of claim 11, wherein said composition further comprises at least one pharmaceutically acceptable carrier.

13. The method of claim 11, wherein said flavor masking agent is a gluconate salt.

14. The method of claim 11, wherein said sweetener is sucralose.

15. The method of claim 1, wherein said branched chain amino acid is administered for at least 10 consecutive days.

16. The method of claim 1, wherein said method comprises administering at least about 60 g of valine, leucine, and isoleucine per day.

17. The method of claim 11, wherein said composition comprises valine, leucine, isoleucine, a flavor masking agent, and a sweetener in a ratio of about 1:1:1:1.3:0.9.

18. The method of claim 11, further comprising administering acetate to said subject.

19. The method of claim 18, wherein said acetate is glyceryl triacetate (GTA).
